(19) United States
(12) Patent Application Publication
(10) Pub. No.: US 2004/0033238 A1
(43) Pub. Date: Feb. 19, 2004

(54) Selectable genetic marker for use in pasteurellaceae species

(76) Inventors: Martha H. Mulks, Williamston, MI (US); Paul R. Martin, Sun Lakes, AZ (US)

Correspondence Address: MCLEOD & MOYNE, P.C., 2190 COMMONS PARKWAY, OKEMOS, MI, 48864, US

(21) Appl. No.: 10/296,921

(22) PCT Filed: Nov. 8, 2001
(86) PCT No.: PCT/US01/46804

(51) Int. Cl. A61K 39/02; C12N 1/21

(52) U.S. Cl. 424/200.1; 435/252.3

(57) ABSTRACT

The present invention provides a nucleic acid encoding nicotinamide phosphioribosyltransferase (NadV) from a V-factor independent bacterium and provides methods for using the gene as a selection marker for constructing recombinant bacteria from V-factor dependent bacteria The method is an improvement over methods which rely on nucleic acids which confer antibiotic resistance for constructing recombinant bacteria. Methods for constructing attenuated recombinant (Actinobacillus pleuropneumoniae ) using the selection method of the present invention are also provided.

### CROSS-REFERENCE TO RELATED APPLICATION

This application claims priority to Provisional Application No. 60/246,950, which was filed Nov. 10, 2000.

### STATEMENT REGARDING FEDERALLY SPONSORED RESEARCH OR DEVELOPMENT

This invention was supported by U.S. Department of Agriculture CREES Grants 96-01855 and 98-02202. Therefore, the U.S. Government has certain rights in this invention.

Reference to a “Computer Listing Appendix submitted on a Compact Disc”

Not Applicable.

### BACKGROUND OF THE INVENTION

(1) Field of the Invention

The present invention relates to a nucleic acid encoding a nicotinamide phosphoribosyltransferase (NadV) from a bacteria of the Pasteurellaceae family which is an enzyme in the biochemical pathway for the biosynthesis of nicotinamide adenine dinucleotide (NAD) from nicotinamide. Introducing the nucleic acid encoding the NadV into an EAD-dependent microorganism enables the NAD-dependent microorganism to grow in a medium that does not contain AD. The present invention also relates to a method for selecting recombinant microorganisms which uses the nucleic acid encoding the NadV as a selective marker. In particular, the present invention relates to a method for making recombinant bacteria of the Pasteurellaceae family, in particular Actinobacillus pleuropneumoniae, which uses the nucleic acid encoding the NadV as a selective marker for selecting the recombinant. The method is also useful for facilitating the construction of recombinant bacteria of the Pasteurellaeae family, in particular Actinobacillus pleuropneumoniae, for use in vaccines.

(2) Description of Related Art

In construction of genetically defined mutants of bacteria, it is often necessary to replace the gene to be deleted or modified with a marker gene that confers a selective growth a vantage on the genetically-defined recombinant. This is to ensure that it is possible to identify and select the genetically-defined recombinant from the background of unmodified bacteria. The simplest method is to use a gene encoding antibiotic resistance. However, marker genes that confer antibiotic resistance are not permitted in genetically-defined mutants intended for use in vaccines. At present, there are no reliable methods for constructing genetically-defined mutants of some species of bacteria such as Actinobacillus pleuropneumoniae.

For example, U.S. Pat. No. 5,849,305 to Briggs et al., discloses a method for constructing attenuated Pasteurella haemolytica vaccines in which a portion of the aroA gene is disrupted with a gene that confers antibiotic resistance to the attenuated bacteria; U.S. Pat. No. 5,925,354 to Fuller et al. discloses a method for constructing attenuated Actinobacillus pleuropneumoniae vaccines in which one or more genes of the riboflavin operon are disrupted with a gene that confers antibiotic resistance to the attenuated bacteria; U.S. Pat. No. 6,013,266 to Segers et al discloses a method for constructing attenuated A. pleuropneumoniae vaccines in which the apxIV gene is disrupted; and U.S. Pat. No. 6,180,112 to Highlander et al. discloses a method for constructing attenuated P. haemolytica vaccines in which a portion of the leukotoxin gene is disrupted with a gene that confers antibiotic resistance to the attenuated bacteria. While attenuated bacteria can be constructed using the above methods, because the attenuated bacteria contain a gene that confers antibiotic resistance, the attenuated bacteria cannot be used as a vaccine unless the gene conferring antibiotic resistance is removed. Selection of bacteria that are no longer resistant to antibiotic is difficult to perform. Therefore, there is a need for non-antibiotic selectable marker genes which can be used to construct genetically defined mutant of bacteria for use as vaccines.

Bacteria, like other organisms, are able to synthesize de novo some necessary metabolites while other metabolites need to be provided exogenously. For example, nicotinamide adenine dinucleotide (NAD) is a critical cofactor required for energy metabolism and many oxidation-reduction reactions in both prokaryotic and eukaryotic cells. In many bacterial species, synthesis of NAD occurs de novo via quinolinic acid (Cynamon et al., J. Gen. Microbiol. 134(Pt. 10): 2789-99 (1988); Foster et al., Microbiol. Rev. 44(1): 83-105 (1980)). NAD can also be synthesized by a pyridine nucleotide salvage pathway via nicotinic acid (NA) (Cynamon et al., J. Gen. Microbiol. 134(Pt. 10): 2789-99 (1988); Foster et al., Microbiol. Rev. 44(1): 83-105 (1980)). However, members of the family Pasteurellaceae do not possess either of these pathways for NAD biosynthesis. These bacterial species must acquire this essential nutrient from their environment either as NAD directly, or from a limited number of precursors (Niven and O'Reilly, Intl. J. Syst. Bacteriol. 40(1): 1-4 (1990); O'Reilly and Niven, J. Gen. Microbiol. 132(Pt 3): 807-18 (1986)). This pyridine nucleotide requirement has been historically important in the identification and classification of members of the Pasteurellaceae, with species requiring an NAD supplement for growth in Vitro described as “V-factor dependent” (Kilian, J. Gen. Microbiol. 93(1): 9-62 (1976); Kilian and Biberstein, In Bergey's Manual of Systematic Bacteriology, Vol. 1. Krieg and Holt (Ed.) The Williams and Wilkins Co., Baltimore, Md., pp. 558-575 (1984)). In V-factor dependent species, the pyridine nucleotide source must possess an intact pyridine-ribose bond and the pyridine-carbonyl group must be amidated; therefore, nicotinamide mononucleotide (NMN) and nicotinamide riboside (NR) can function as V-factor, but quinolinic acid (QA), nicotinic acid (NA), nicotinic acid mononucleotide (NAMN), and nicotinamide (NAm) can not (Cynamon et al., J. Gen. Microbiol. 134 (Pt. 10): 2789-99 (1988); O'Reilly and Niven, J. Gen. Microbiol. 132(Pt 3): 807-18 (1986)).

The ability to us nicotinamide (NAm) as a precursor for NAD biosynthesis has been shown to differentiate V-factor dependent from V-factor independent strains (O'Reilly and Niven, Can. J. Microbil. 32(9): 733-7 (1986)). Haemophilus haemoglobinophilus, which is V-factor independent, synthesizes the enzyme nicotinamide phosphoribosyltransferase, which converts NAm to NMN and allows the use of NAm as a source of pyridine nucleotide (Kasarov and Moat, Biochim. Biophys. Acta 320(2): 372-8 (1973)) (FIG. 1). Since NAm is available in most complex bacteriologic media, bacteria that can utilize NAm are V-factor independent.

In many species of Pasteurellaceae defined as V-factor dependent, V-factor independent variants have been identified. These include strains of Actinobacillus pleuropneumoniae, which causes pleuropneumoniae in swine (Pohl et al., Intl. J. Syst. Bacteriol. 11(3): 510-514 (1983)); Haemophilus paragallinarum, which causes fowl choryza (Bragg et al., J. Vet. Res. 60(2): 147-52 (1993); Miflin et al., Avian Dis. 39(2): 304-8 (1995)); H. parainfluenzae, which can cause pneumonia and meningitis in humans (Gromkova and Koornhof, J. Gen. Microbiol. 136(Pt 6): 1031-5 (1990)); and H. ducreyi, which cause the sexually transmitted disease chancroid in humans (Windsor et al., Med. Microbiol. Lett. 2: 159-167 (1993); Windsor et al., J. Genl. Microbial. 137(Pt 10): 2415-21(1991)). In H. parainfluenzae, H. paragallinarum, and H. ducreyi, V-factor independence has been shown to be encoded on a plasmid (Bragg et al., J. Vet. Res. 60(2): 147-52 (1993); Windsor et al., J. Genl. Microbial. 137 (Pt 10): 2415-21(1991); Windsor et al., Intl. J. Syst. Bacteriol. 43(4): 799-804(1993)). However, because V-factor independence has been presumed to be encoded by more than one gene (Windsor et al., J. Genl. Microbial. 137 (Pt 10): 2415-21 (1991); Windsor et al., Intl. J. Syst. Bacteriol. 43(4): 799-804 (1993)), a selection method for recombinant bacteria based on V-factor independence is impractical Even though Holloway de Corsier (Ph.D. Dissertation. University of Berne, Berne, Switzerland, (1994)) reported that in A. pleuropneumoniae V-factor in dependence may be conferred by a chromosomal gene, to date, not a single gene related to V-factor independence has been identified or isolated.

In light of the above, there remains a need for methods for constructing recombinant bacteria that do not rely on antibiotic resistance for selection. In particular, a need remains for methods for constructing live attenuated bacterial vaccines that do not rely on antibiotic resistance for selection.

### SUMMARY OF THE INVENTION

The present invention provides a nucleic acid encoding nicotinamide phosphoribosyltransferase (NadV) from a V-factor independent bacterium and provides methods for using the gene as a selection marker for constructing recombinant bacteria from V-factor independent bacteria. The method is an improvement over methods which rely on nucleic acids which confer antibiotic resistance for constructing recombinant bacteria. Methods for constructing attenuated recombinant Actinobacillus pleuropneumoniae using the selection method of the present invention are also provided.

Therefore, the present invention provides an isolated nucleic acid encoding a nicotinamide phosphoribosyl transferase (NadV) from an organism which confers V-factor independence when transformed into a V-factor dependent Pasteurellaceae spp. or strain.

In a particular embodiment, the organism is a microorganism selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haeinphilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp.

In a further embodiment, the isolated nucleic acid encodes the NadV from Haemophilus ducreyi which is ATCC 27722.

In a further still embodiment, the isolated nucleic acid is operably linked to a heterologous promoter.

In an embodiment further still, the NadV comprises an amino acid sequence selected from the group consisting of SEQ ID NO:2, SEQ ID NO:3, SEQ ID NO:4, SEQ II NO:5, SEQ ID NO:6, SEQ ID NO:7, SEQ ID NO:8, SEQ ID NO:9, and SEQ ID NO:10 and including amino acid sequence variants thereof which do not abrogate the ability of the NadV to confer V-factor independence to a V-factor dependent bacterium. Preferably, wherein the isolated nucleotide sequence encoding the NadV comprises the nucleic acid sequence set forth in SEQ ID NO:1 and including nucleic acid sequence variants thereof which do not abrogate the ability of gene to confer V-factor independence to a V-factor dependent bacterium.

The present invention also provides a plasmid comprising a nucleotide sequence encoding a nicotinamide phosphoribosyl transferase (NadV) which comprises the nucleic acid sequence set forth in SEQ ID NO:1, including sequence variants thereof which do not abrogate the ability of gene to confer V-factor independence to a V-factor dependent bacterium, and wherein expression of the gene encoding the NadV is under control of a heterologous promoter. Preferably, the plasmid is an E. coli-Pasteurellaceae spp. shuttle vector or a plasmid for homologous recombination.

Further still, th present invention provides a method for constructing a genetically defined recombinant Pasteurellaceae spp. comprising (a) providing a gene encoding nicotinamide phosphoribosyl transferase (NadV) in a plasmid, preferably a suicide plasmid, that targets a genomic nucleic acid sequence in a V-factor dependent Pasteurellaceae spp.; (b) transforming the V-factor dependent Pasteurellaceae spp. with the vector wherein the genomic nucleic acid sequence in the Pasteurellaceae spp. is replaced or partially replaced with the gene encoding the NadV, which renders the Pasteurellaceae spp. capable of growing in media free and nicotinamide adenine dinucleotide (NAD) and nicotinamide mononucleotide (NMN); and (c) selecting the genetically defined recombinant in media free of NAD and NMN wherein the recombinant Pasteurellaceae spp. comprises the gene encoding the NadV in place of the genomic nucleic acid sequence.

In a particular embodiment of the method, the Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi

In a further embodiment of the method, the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella Synechocystis spp.

In an embodiment further still of the method, thee gene encoding the NadV is from Haemophilus ducreyi deposited as ATCC 27722. In a preferred embodiment, the gene encoding the NadV is operably linked to a heterologous promoter. In a further preferred embodiment, the gene encoding the NadV comprises a nucleic acid sequence with the nucleic acid sequence set forth in SEQ ID NO:1 and including nucleic acid sequence variants thereof which do not abrogate the ability of gene to confer V-factor independence to a V-factor dependent bacterium.

In an embodiment of the method further still, the genomic nucleic acid sequence comprises one or more genes that are necessary for survival of the Pateurellaceae spp. in vivo Preferably, the genomic nucleic acid sequence comprises one or more genes selected from the group consisting of genes for riboflavin biosynthesis, genes for aromatic amino acid biosynthesis, genes for isoleucine, leucine, and valine biosynthesis, genes for virulence factor, and combinations thereof. In particular, wherein the genomic nucleic acid sequence encodes a gene selected from the group consisting of ribA, ribB, ribH, aroA, ilvI, lktC, apxIV, and combinations thereof.

The present invention further provides a genetically defined recombinant Pasteurellaceae spp. comprising a gene encoding nicotinamide phosphoribosyl transferase (NadV) inserted into a genomic nucleic acid sequence of a V-factor dependent Pasteurellaceae spp. wherein the gene encoding the NadV enables the recombinant Pasteurellaceae spp. to grow in media free of nicotinamide adenine dinucleotide and nicotinamide mononucleotide.

In a particular embodiment of the genetically defined recombinant, the V-factor dependent Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

In a further embodiment of the genetically defined recombinant, the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella aemolytica, Pasteurella multocida, Shewanella putrefaciens, and Synechocystis spp. In a preferred embodiment, the gene encoding the NadV is from Haemophilus ducreyi deposited as ATCC 27722. It is further preferable that the gene encoding the NadV is operably linked to a heterologous promoter. In a further still preferred embodiment, it is preferable that the gene encoding the NadV comprises a nucleic acid sequence with the nucleic acid sequence set forth in SEQ ID NO:1 and including nucleic acid sequence variants thereof which do not abrogate the ability of gene to confer V-factor independence to a V-factor dependent bacterium.

In further embodiment of the genetically defined recombinant, the genomic nucleic acid sequence comprises one or more gene that are necessary for survival of the Pasteurellaceae spp. in vivo. Preferably, the genomic nucleic acid sequence comprises one or more genes selected from the group consisting of genes for riboflavin biosynthesis, genes for aromatic amino acid biosynthesis, genes for isoleucine and valine biosynthesis, genes for virulence factor, and combinations thereof. In particular, wherein the genomic nucleic acid sequence encodes a gene selected from the group consisting of ribA, ribB, ribH, aroA, ilvI, lktC, apxIV, and combinations thereof.

In a further embodiment of the genetically defined recombinant, the NadV comprises an amino acid sequence selected from the group consisting of SEQ ID NO: 2, SEQ ID NO: 3, SEQ ID 04, SEQ ID NO: 5, SEQ ID NO: 6, SEQ ID NO: 7, SEQ ID NO: 8, SEQ ID NO: 9, and SEQ ID is NO:10.

The present invention further provides a vaccine for immunizing a host against a Pasteurellaceae spp. comprising a recombinant V-factor independent Pasteurellaceae spp. comprising a gene encoding nicotinamide phosphoribosyl transferase (NadV) inserted into a genomic nucleic acid sequence of a V-factor dependent Pasteurellaceae spp. or strain wherein the gene encoding the nadV disrupts expression of one or more genes encoded by the genomic nucleic acid sequence to produce the recombinant V-factor independent Pasteurellaceae spp. or strain a pharmaceutically acceptable carrier in amount effective to produce an immune response.

In a particular embodiment of the vaccine, the V-factor dependent Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

In a further embodiment of the vaccine, the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp. In a preferred embodiment, the gene encoding the NadV is from Haemophilus ducreyi deposited as ATCC 27722. It is further preferable that the gene encoding the NadV is operably linked to a heterologous promoter. In a further still preferred embodiment, it is preferable that the gene encoding the NadV comprises a nucleic acid sequence wit the nucleic acid sequence set forth in SEQ ID NO:1 nd including nucleic acid sequence variants thereof which do not abrogate the ability of gene to confer V-factor independence to a V-factor dependent bacterium.

In a further embodiment of the vaccine, the genomic nucleic acid sequence comprises one or more genes that are necessary for survival of the Pasteurellaceae spp. in viva. Preferably, the genomic nucleic acid sequence comprises one or more genes selected from the group consisting of genes for riboflavin biosynthesis, genes for aromatic amino acid biosynthesis, genes for isoleucine, leucine, and valine biosynthesis, genes for a virulence factor, and combinations thereof. In particular, wherein the genomic nucleic acid sequence encodes a gene selected from the group consisting of ribA, ribB, ribH, aroA, ilvI, lktC, apxIV, and combinations thereof.

In a further embodiment, the vaccine contains an adjuvant. While in one embodiment the recombinant Pasteurellaceae spp. is inactivated, in a preferred embodiment, the recombinant Pasteurellaceae spp. is live.

In a further embodiment of the vaccine, the NadV comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

The present invention further provides a method for immunizing a host against a Pasteurellaceae spp. comprising administering to the host an effective dose of a vaccine comprising a recombinant V-factor independent Pasteurellacea spp. comprising a gene encoding nicotinamide phosphribosyl transferase (NadV) inserted into a genomic nucleic acid sequence of a V-factor dependent Pasteurellaceae spp. or strain wherein the gene encoding the NadV disrupts expression of one or more genes encoded by the genomic nucleic acid sequence to produce the recombinant V-factor independent Pasteurellaceae spp. or strain, in a pharmaceutically acceptable carrier.

In a particular embodiment of the method, the V-factor dependent Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

In a further embodiment of the method, the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp. In a preferred embodiment, the gene encoding the NadV is from Haemophilus ducreyi deposited as ATCC 27722. It is further preferable that the gene encoding the NadV is operably linked to a heterologous promoter. In a further still preferred embodiment, it is preferable that the gene encoding the NadV comprises a nucleic acid sequence with the nucleic acid sequence set forth in SEQ ID NO:1 and including nucleic acid sequence variants thereof which do not abrogate the ability of gene to confer V-factor independence to a V-factor dependent bacterium.

In a further embodiment of the method, the genomic nucleic acid sequence comprises one or more genes that are necessary for survival of the Pasteurellaceae spp. in vivo Preferably, the genomic nucleic acid sequence comprises one or more genes selected from the group consisting of genes for riboflavin biosynthesis, genes for aromatic amino acid biosynthesis, genes for isoleucine and valine biosynthesis, genes for a virulence factor, and combinations thereof. In particular, wherein the genomic nucleic acid sequence encodes a gene selected from the group consisting of ribA, ribB, ribH, aroA, ilvI, lktc, apxIV, and combinations thereof.

In a further embodiment of the method contains.

In a further embodiment of the method contains an adjuvant. While in one embodiment the recombinant Pasteurellaceae spp. is inactivated, in a preferred embodiment, the recombinant Pasteurellaceae spp. is live.

The present invention further provides a method for reducing the cost of growing a V-factor dependent Pasteurellaceae spp. comprising (a) transforming the V-factor dependent Pasteurellaceae spp. with a gene encoding a nicotinamide phosphoribosyl transferase (NadV) to produce a recombinant Pasteurellaceae spp. wherein the gene encoding the NadV renders the V-factor dependent Pasteurellaceae spp. V-factor independent; and (b) growing the recombinant Pasteurellaceae spp. in media free of nicotinamide adenine dinucleotide (AD) and nicotinamide mononucleotide (NMN) which reduces the cost of growing the V-factor dependent Pasteurellaceae spp. The present invention further provides a method for growing a V-factor dependent Pasteurellaceae spp. in a medium free of nicotinamide adenine dinucleotide (NAD) and nicotinamide mononucleotide (NMN) comprising (a) transforming the V-factor dependent Pasteurellaceae spp. with a gene encoding a nicotinamide phosphoribosyl transferase (NadV) to produce a recombinant Pasteurellaceae spp. wherein the gene encoding the NadV renders the V-factor dependent Pasteurellaceae spp. V-factor independent; and (b) growing the recombinant Pasteurellaceae spp. in the medium free of NAD and NMN which reduces the cost of growing the V-factor dependent Pasteurellaceae spp.

In a further embodiment of the above method, the Pasteurellaceae spp. is selected from the group consisting of Actinobacillus pleuropneumoniae, paragallinarum, Haemophilus parainfluenzae, Haemophilus parasuis, Haemophilus ducreyi.

In an embodiment further still of either of the above methods, the gene encoding the NadV is from a bacterium selected from he group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella aemolytica, Pasteurella multocida, Shewanella putrefaciens, and Synechocystis spp.

In an embodiment further still of either of the above methods, the gene encoding the NadV is from Haemophilus ducreyi deposited as ATCC 27722. Preferably, the gene encoding the NadV is operably linked to a heterologous promoter. In a further embodiment, the gene encoding the NadV comprises a nucleic acid sequence with the nucleic acid sequence set forth in SEQ ID NO:1.

In a further embodiment of either of the above methods, the gene encoding the NadV is on a plasmid or the gene encoding the NadV replaces a portion of a genomic nucleic acid sequence of the V-dependent Pasteurellaceae spp. In a further embodiment, the genomic nucleic acid sequence encodes one or more genes necessary for survival of the Pasteurellaceae spp. in vivo. In particular, wherein the genomic nucleic acid sequence encodes one or more genes selected from the group consisting of genes for riboflavin biosynthesis, genes for aromatic amino acid biosynthesis, genes for isoleucine and valine biosynthesis, genes for a virulence factor, and combinations thereof or more particularly, wherein the genomic nucleic acid sequence encodes a gene selected from the group consisting of ribA, ribB, ribH, aroA, ilvI, lktC, apxIV, and combinations thereof.

In a further embodiment of any one of the above methods, the NadV comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Finally, the present invention provides an isolated nucleic acid which encodes a protein that confers V-factor independence to a V-factor dependent bacteria when transformed into the V-factor dependent bacteria selected from the group consisting of SEQ ID NO:1, SEQ ID NO:13, SEQ ID NO:14, SEQ ID NO:15, SEQ ID NO:16, and SEQ ID NO:19.

### OBJECTS

Therefore, it is an object of the present invention to provide a method for constructing genetically-defined attenuated bacteria that does not rely upon antibiotic resistance for recovering the attenuated bacteria.

It is a further object of the present invention to provide vaccines that are made according to the method of the present Invention.

It is a further object of the present invention to provide a gene encoding nicotinamide phosphoribosyl transferase which is used in the method of the present invention to construct genetically-defined attenuated bacteria.

These and other objects of the present invention will become increasingly apparent with reference to the following drawings and preferred embodiments.

### DESCRIPTION OF THE DRAWINGS

FIG. 1 shows the biochemical pathway for the biosynthesis of nicotinamide adenine dinucleotide (NAD) as found in the family Pasteurellaceae. NAD-dependent species lack the ability to convert nicotinamide (NAm) to nicotinamide mononucleotide (NMN)

FIG. 2 shows subclones of pNAD1 constructed in the E. coli-A. pleuropneumoniae shuttle vector pGZRS18 (West et al., Gene. 160(1): 81-6 (1995)). The location of the NadV gene is indicated with an arrow. Plasmids pGZNAD1, pGZNAD7, an pGZNAD8 were constructed using the restriction sites shown. Plasmid pGZNAD9 was constructed using synthetic primers to PCR amplify the nadV gene. The ability of these clones to confer NAD-independence to A. pleuroneumoniae is indicated in the right-hand column. Restriction sites are: A, AvaI; B, BamHI; E, EcoRI; and P, PstI.

FIG. 3 shows the alignment of the predicted NadV amino acid sequence with homologues found in other species. Black shaded regions indicate residues that are identical in the majority species. Gray shaded regions indicate residues that are functionally conserved in the majority of species. Species abbreviations include: Aact, Actinobacillus actinomycetemcomitans; Pasteurella multocida; Drad Deinococcus radiodurans; Syn, Synechocystis; Mgen, Mycoplasma genitalium; Mpne, M. pneumoniae; Sput, Shewanella putrefaciens; Hduc, Haemophilus ducreyi; Hum, human PBEF. The alignment was obtained using the Pileup program from the Genetics Computer Group package (Genetics Computer Group. Program Manual for the Wisconsin Package, 10^{th} Ed Genetics Computer Group, Madison, Wis., (1999)).

FIG. 4 shows the incorporation of ¹⁴C-nicotinamide into NMN. NAm-PRTase assays were performed with ¹⁴C-nicotinamide as substrate and incorporation of radiolabel into NM followed with time.

The dark bars are A. pleuroneumoniae/pGZNAD9 and the light bars are A. pleuropneumoniae/pGZRS18.

FIG. 5 shows a diagram of the relevant region of plasmid pC18KnadV which contains a gene expression cassette containing the NadV gene operably linked at the 5′ end to the kanamycin promoter region in plasmid pUC18. The kanamycin promoter is operable in Pasteurellaceae spp. KanP is the kanamycin promoter region operably linked to the nadV gene. The restriction enzyme sites are as follows: E is EcoRI, B is BamHI, Pst is PstI, Nco is NcoI, Sph is SphI, and Hind is HindIII.

FIG. 6 shows a diagram of the relevant region of plasmid pC18KanNad which contains the NadV and kanamycin double-selection gene expression cassette in pUC18. Kan^{R} is the kanamycin gene expression cassette from pUC4K, KanP is the kanamycin promoter region operably linked to the nadV gene. The restriction enzyme sites are as follows: is EcoRI, B is BamHI, Pst is PstI, Nco is NcoI, Sph is SphI, and Hind is HindIII.

FIG. 7 shows a diagram of the relevant portion of plasmid pilvI5′3′ which contains the 5′ and 3′ end DNA fragments of the A. pleuropneumoniae ilvI gene in pUC18. The restriction enzyme sites are as follows: Eco is EcoRI, B is BamHI, Sac is SacI, Kpn is KpnI, Sph is SphI, and H is HindIII.

FIG. 8 shows a diagram of the relevant portion of plasmid pC18ilvKanNad which contains the nadV and kanamycin double-selection gene expression cassette inserted into the BamHI site of pilvI5′3′. Kan^{R} is the kanamycin gene expression cassette from pUC4K and KanP-nadV is the NadV gene express on cassette of pC18KanNad. The restriction enzyme sites are as follows: E is EcoRI, B is BamHI, Pst is PstI, Nco is NcoI, Sph is SphI, and Hind is HindIII.

FIG. 9 shows a diagram of the relevant portion of plasmid pTF66-nadV which contains the nadV gene expression cassette of plasmid pC18KnadV inserted between the ClaI and NdeI sites of pTF66. KanP is the kanamycin promoter region operably linked to the nadV gene. ribB(−3′) is the ribB gene of the riboflavin operon with about 150 bp of he 3′ end deleted and ribH is the ribH gene of the riboflavin operon. The restriction enzyme sites are as follows: E is EcoRI and Hind is HindIII.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The present invention provides the enzyme nicotinamide phosphoribosyltransferase (NadV) from H. ducreyi and an isolated DNA comprising the nadV gene encoding the NadV protein. Because members of the family Pasteurellaceae are classified in part by whether they require a nicotinamide adenine dinucleotide (NAD) supplement for growth in bacterial media (V-factor dependent) or not (V-factor independent), the present invention also provides a method, which uses the nadV gene as a selectable marker, for constructing recombinant bacteria from V-factor dependent bacteria. Recombinant bacteria are selected by their ability to grow in media without NAD (V factor independence). The method is an improvement over current methods for constructing recombinant bacteria which rely on genes that encode antibiotic resistance factors as selectable markers for isolating recombinant bacteria.

As shown in Example 1, V-factor dependence or independence is determined by the lack or presence of the nadV gene. Species of Pasteurellaceae which are V-factor dependent have been identified in strains such as Actinobacillus pleuropneumoniae, Actinobacillus suis, Haemophilus influenzae, Haemophilus paragallinarum, Haemophilus parainfluenzae Haemophilus parasuis, Haemophilus ducreyi.

In general, present methods for constructing recombinant bacteria rely or introducing an antibiotic resistance gene into the bacteria to enable the recombinant bacteria to be selected from non-recombinant bacteria. While the present methods are efficient for producing recombinant bacteria, to use the recombinant bacteria as a vaccine, the antibiotic gene has to be removed from the recombinant bacteria. Isolating recombinant bacteria with the antibiotic gene removed is difficult because there is no good selection method for isolating the recombinant bacteria with the antibiotic gene removed.

In contrast, because the method of the present invention uses the nadV gene instead of a gene conferring antibiotic resistance as the selectable marker for isolating recombinant bacteria, recombinant bacteria constructed using the method of the present invention can be used in vaccines without having to remove the selectable marker from the recombinant bacteria. Furthermore, in the case of growing V-factor dependent bacteria for vaccines, the media must be supplemented with NAD. Supplementing media for growing V-factor dependent bacteria with NAD for vaccine production is expensive. Because recombinant bacteria containing the nadV are V-factor independent, the present invention enables the recombinant bacteria to be grown at less cost than the non-recombinant V-factor dependent bacteria.

The nicotinamide phosphoribosyltransferase (NadV) of the present invention comprises the amino acid sequence set forth in SEQ ID NO:2, which is encoded by the nadV gene comprising the nucleotide sequence set forth in SEQ ID NO:1. The nadV is isolatable from H. ducreyi and has the ability to confer V-factor independence to V-factor dependent bacteria when transformed into the V-factor dependent bacteria. The H. ducreyi containing the plasmid from which the nucleic acid comprising SEQ ID NO:1 was isolated is commercially available from the American Type Culture Collection, 10801 University Boulevard, Manassas, Virginia, as ATCC 27722.

The present invention comprises the NadV having the amino acid sequence of SEQ ID NO:2 and mutants thereof which are encoded by the nadV having the nucleic acid sequence of SEQ ID NO:1 and mutants thereof. As used herein, “mutants thereof” refers to mutations, modifications, or variations in the amino acid sequence of the NadV or the nucleotide sequence encoding the NadV which differ from the amino acid or nucleotide sequences provide herein but which do not * abrogate the ability of the NadV to confer V-factor independence to V-factor dependent bacteria.

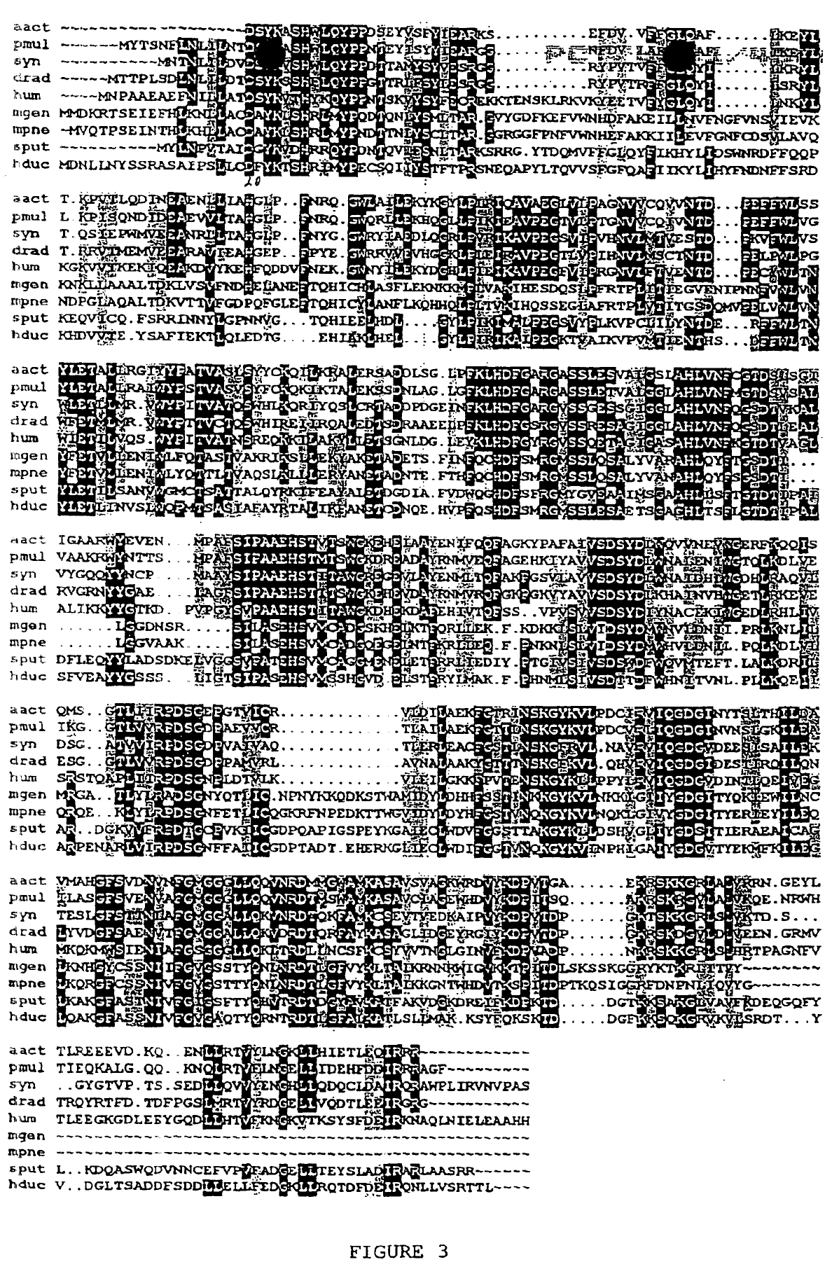

The NadV of the present invention further includes proteins which have the amino acid sequence set forth in SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and mutants thereof which correspond to the amino acid sequences of open reading frames (ORFs) from Actinobacillus actinomycetemcomitans, Pasteurella multocida, Deinococcus radiodurans, Synechocystis spp., mammalian pre-B cell colony enhancing factor (PBEF) from a human, Mycoplasma genitalium, Mycoplasma pneumoniae, and Shewanella putrefaciens respectively, and mutants thereof. As shown in FIG. 3, the proteins were discovered to have substantial identity to the NadV amino acid sequence of SEQ ID NO:2. The M. pneumoniae protein of SEQ ID NO:9 was reported in GenBank (Accession NO: NP_{—}109735) to have identity to NadV, the P. multocida protein of SEQ ID NO: 4 and the M. genitalium protein of SEQ ID NO:8 were reported in GenBank (Accession Nos: NP_{—}245936 and NP_{—}072697, respectively) to be proteins of unknown function, and the D. radiodurans protein of SEQ ID NO:5 and the Synechocystis spp. protein of SEQ ID NO:6 were reported in GenBank (Accession Nos: NP 294017 and S7702, respectively) to have identity to the mammalian pre-B cell enhancing factor (PBEF) of SEQ ID NO:7.

Because the above proteins have substantial identity to the NadV, the above proteins have the ability to confer V-factor independence to V-factor dependent bacteria. For example, the murine PBEF confers NAD independence to V-factor dependent bacteria. Therefore, the NadV includes not only the NadV and mutants thereof of H. ducreyi but also the above proteins and mutants thereof of A. actinomycetemcomitans, P. multocida, D. radiodurans, Synechocystis spp., M. genitalium, M. pneumoniae, S. putrefaciens, and mammalian PBEF which as shown herein, have substantial identity to the NadV of H. ducreyi and which have the ability to render V-factor dependent bacteria V-factor independent. Furthermore, the nucleotide sequences encoding the NadV homologues of D. radiodurans, Synechocystis spp., M. genitalium, M. pneumoniae, P. multocida, and S. putrefaciens are set forth in SEQ ID NOs:13, 14, 15, 16, and 17, respectively. The nucleotide sequence of mammalian pre-B-cell colony enhancing factor (PBEF) is set forth in SEQ ID NO:19. Thus, the present invention further includes the nucleic acid sequences set forth in SEQ ID NOs:13, 14, 15, 16, 17, and 19, and mutants thereof.

The present invention further provides a positive selection method for making recombinant bacteria. In particular, the NadV and mutants thereof are used in a positive select on method for constructing recombinant bacteria from bacteria which are V-factor dependent. In practice, V-factor dependent bacteria are transformed with a DNA which comprises the nadV using any one of the transformation methods known in the art. The recombinant bacteria in the transformation contain the nadV which renders the recombinant bacteria V-factor independent. However, in any transformation, the transformation produces a mixture of bacteria wherein only a portion of the bacteria are transformed and, therefore, are recombinant bacteria. To select the recombinant bacteria from non-recombinant bacteria, the bacteria mixture is incubates media lacking NAD. In media lacking NAD (e.g., brain-heart infusion broth without NAD) or in a chemically defined media containing Nam but not NAD, only the transformed or recombinant bacteria in the mixture grow. The non-recombinant bacteria in the mixture do not grow. Because NAD is a requirement for growth of V-factor dependent bacteria, the method provides a clean and efficient positive selection method for separating recombinant bacteria from non-recombinant bacteria.

As used herein, recombinant bacteria includes both recombinant bacteria wherein the nadV is integrated into the genome of the bacteria and recombinant bacteria wherein the bacteria have been transformed with a plasmid containing the nadV and the nadV remains on the plasmid, which replicates autonomously in the bacteria. In a preferred embodiment, the NadV or mutant thereof is operably linked to a heterologous promoter that enables expression of the NadV constitutively, e.g., operably linked to the kanamycin gene promoter, or to an inducible promoter, e.g., operably linked to the beta-galactosidase gene promoter.

Examples 4, 5, an 6 provide examples of the selection method of the pr sent invention wherein V-factor dependent Actinobacillus pleuropneumoniae was transformed with a plasmid homology vector containing the NadV operably linked to a kanamycin promoter and flanked by sequences homologous to the ilvI gene or riboflavin genes, respectively, wherein in the transformed bacteria, the nadV is integrated into the Actinobacillus pleuropneumoniae genome by homologous recombination. Selection of the recombinant Actinobacillus pleuropneumoniae, which had been rendered V-factor independent by the NadV, was by the recombinant's ability to grow in media that did not contain NAD.

Examples 1 and 2 provide examples of the selection method of the present invention wherein Actinobacillus pleuropneumoniae is transformed with a plasmid containing the nadV on an E. coli-Actinobacillus pleuropneumoniae shuttle vector wherein the nadV remains on the plasmid which replicates autonomously in the bacteria. In Example 1, expression of the nadV was by a heterologous promoter resident in the plasmid and in Example 2, the nadV was operably linked to the kanamycin promoter. Selection of the recombinant Actinobacillus pleuropneumoniae, which ha been rendered V-factor independent by the nadV, as by the recombinant's ability to grow in media that did not contain NAD.

In a preferred embodiment, recombinant V-factor independent bacteria are constructed from V-factor bacteria such as Actinobacillus pleuropneumoniae by any of the methods well known in the art, e.g., transformation by electroportion or mating between E. coli containing the plasmid and the V-factor dependent bacteria. For example, both methods for making recombinant bacteria are disclosed in the examples and in U.S. Pat. No. 5,849,305 to Briggs et al., U.S. Pat. No. 5,925,354 to Fuller et al., U.S. Pat. No. 6,013,266 to Segers et al., and U.S. Pat. No. 6,180,112 to Highlander et al.

Because the positive selection method of the present invention, which uses the nadV and mutants thereof, is useful for constructing recombinant bacteria from bacteria which are V-factor dependent, the positive selection method of the present invention is useful for constructing recombinant bacteria vaccines. Thus, the present invention further provides recombinant bacteria vaccines and methods for making the recombinant bacteria using the positive selection method of the present invention.

In one embodiment of a recombinant bacteria vaccine and method for making the recombinant bacteria, the recombinant bacteria is made from an attenuated or avirulent V-factor dependent bacteria wherein the nadV or mutant thereof has been inserted into the genome of the attenuated V-factor dependent bacteria or wherein the nadV or mutant thereon is on a plasmid in the attenuated V-factor dependent bacteria. In another embodiment of a recombinant bacteria vaccine and method for making the recombinant bacteria, the recombinant bacteria is made from a virulent V-factor dependent bacteria wherein the nadV or mutant thereof has been inserted into a region of the genome of the virulent V-factor dependent bacteria which attenuates the bacteria or renders the bacteria avirulent. In either embodiment, the recombinant bacteria is rendered V-factor independent by the nadV or mutant thereof. Preferably, the recombinant vaccine is made from a V-factor dependent bacteria from the Pasteurellaceae family.

In a particular embodiment of the recombinant bacteria vaccine, the present invention provides attenuated or avirulent recombinant Pasteurellaceae spp. or strain vaccines and methods for making the attenuated or avirulent recombinant Pasteurellaceae spp. or strain vaccines wherein the nadV or mutant thereof is inserted into at least one essential or virulence gene in the genome of a V-factor dependant Pasteurellaceae spp. or strain so as to disrupt expression of the essential or virulence gene thereby rendering the V-factor dependent Pasteurellaceae spp. or strain attenuated or avirulent. Because the nadV or mutant thereof renders the Pasteurellaceae spp. or strain V-factor independent, the nadV or mutant thereof inserted into at least one essential or virulence gene enables the attenuated or avirulent recombinant Pasteurellaceae spp. or strain to be isolated from parental V-factor dependent Pasteurellaceae spp. or strain.

Preferably, the nadV or mutant thereof replaces or partially replaces a segment of DNA in the genome of the Pasteurellaceae spp. or strain which encodes one or more enzymes necessary for growth of the Pasteurellaceae spp. or strain or which encodes a virulence factor. For example, an attenuated or avirulent Pasteurellaceae sp. or strain is made wherein the NadV or mutant thereof replaces or partially replaces one or more genes in the aromatic amino acid biosynthetic pathway, e.g., the aroA gene as taught in U.S. Pat. No. 5,849,305 to Briggs et al., the NadV replaces or partially replaces the lktc gene encoding leukotoxin as taught in 0.5. U.S. Pat. No. 6,180,112 to Highlander et al., the nadV replaces or partially replaces the apxIV gene as taught in U.S. Pat. No. 6,013,266 to Segers et al., the NadV replaces or partially replaces one or more genes in the riboflavin synthesis pathway as taught in U.S. Pat. No. 5,925,354 to Fuller et al., or the nadV replaces or partially replaces an acetohydroxy acid synthase gene such as the ilvI gene involved in the biosynthesis of isoleucine and valine (Fuller et al., Microb. Pathol. 27(5): 311-327 (1999)) as taught herein. Preferably, the nadV replaces or partially replaces the ilvI gene in the isoleucine and valine biosynthesis pathway or one or more genes in the riboflavin synthesis pathway.

The route of administration for the attenuated or avirulent and V-factor independent recombinant Pasteurellaceae spp. or strain vaccine of the present invention includes, but is not limited to, intramuscular, intraperitoneal, intradermal, subcutaneous, intravenous, intra-arterial, intra-ocular, and trans-dermal or by inhalation, ingestion, or suppository. The preferred routes of administration include intramuscular, intraperitoneal, intradermal, and subcutaneous injection, or by inhalation. Most preferably, the attenuated or avirulent and V-factor independent recombinant Pasteurellaceae spp. or strain vaccine is injected intramuscularly. The attenuated or avirulent and V-factor independent recombinant Pasteurellaceae spp. or strain vaccine can be administered by means including, but not limited to, syringes, needle-less injection devices, or microprojectile bombardment gene guns.

The attenuated or avirulent and V-factor independent recombinant Pasteurellaceae spp. or strain vaccine of the present invention is formulated in a pharmaceutically accepted carrier according to the mode of administration to be used. In cases where intramuscular injection is preferred, a sterile water or isotonic formulation is preferred. Generally, additives for isotonicity can include sodium chloride, dextrose, mannitol, sorbitol, and lactose. In particular cases, isotonic solutions such as phosphate buffered saline are preferred. The formulations can further provide stabilizers such as gelatin and albumin. In some embodiments, a vaso-constriction agent is added to the formulation. An adjuvant which can be used for the vaccine is EMULSIGEN (MVP Labs, Ralston, Nebr.), which is a paraffin oil in water emulsion, which can be used in food animals. Freund's Incomplete Adjuvant, which is 15 percent by weight mannide monooleate and 85% paraffin oil, available from Difco, Detroit, Mich., can be used in non-food (i.e. laboratory animals). The adjuvants aid in slowly releasing the vaccine into the animal and can potentiate the immune response. Any commercial oil emulsion adjuvants can be used such as vitamin E. The most preferred carrier is sterile water or an aqueous saline solution, particularly when the vaccinee is a human.

The pharmaceutical preparation according to the present invention are provided sterile and pyrogen free. However, it is well known by those skilled in the art that the preferred formulations for the pharmaceutically accepted carrier which comprise the attenuated or avirulent nd V-factor independent recombinant Pasteurellaceae spp or strain vaccine of the present invention are the pharmaceutical carriers approved in the regulations promulgated by the United States Department of Agriculture, or equivalent government agency in a foreign country such as Canada or Mexico, for vaccines intended for veterinary applications. Therefore, a pharmaceutically accepted carrier for commercial production of the attenuated or avirulent and V-factor independent recombinant Pasteurellaceae spp. or strain vaccine of the present invention is a carrier that is already approved or will at some future date be approved by the appropriate government agency in the United States of America or foreign country.

Inoculation of the vaccinee with the attenuated or avirulent and V-factor independent recombinant Pasteurellaceae spp. or strain vaccine is preferably by a single vaccination. In another embodiment of the present invention, the vaccinee is subjected to a series of vaccinations to produce a full, broad immune response. When the vaccinations are provided in a series, the vacinations can be provided between about 24 hours apart to two weeks or longer between vaccinations. In certain embodiments, the vaccinee is vaccinated at different sites simultaneously.

While the above methods for constructing recombinant bacteria and the vaccines have been described herein using the NadV and mutants thereof of H. ducreyi, the present invention is not limited to the NadV and mutants thereof of H. ducreyi. The present invention further includes the above methods for constructing recombinant bacteria and vaccines using genes encoding the nadV homologues which have the amino acid sequences set forth in SEQ ID NO:3, SEQ ID NO:4, SEQ ID NO:5, SEQ ID NO:6 SEQ ID NO:7, SEQ ID NO:8, SEQ ID. NO:9, and SEQ ID NO:10, ad mutants thereof, which correspond to the amino acid sequences of open reading frames (ORFs) from Actinobacillus actinomycetemcomitans, Pasteurella multocida, Deinococcus radiodurans, Synechocystis spp., mammalian pre-B cell enhancing factor (PBEF), Mycoplasma genitalium, Mycoplasma pneumoniae, and Shewanella putrefaciens, respectively, and mutants thereof.

The present invention further includes the above methods for constructing recombinant bacteria and vaccines using genes encoding the nadV homologue using a nucleic acid selected from the group consisting of SEQ ID NO:1, SEQ ID NO:13, SEQ ID NO:14, SEQ ID NO:15, SEQ ID, NO:16, SEQ ID NO:17, SEQ ID NO:19 (Human PBEF; GenBank Accession No. U02020), SEQ ID NO:20, and SEQ ID NO:21, and mutants thereof. SEQ ID NO:20 is a nucleic acid from Cyprinus carpio (common carp; GenBank Accession No. AB027712) in which codons 29 to 2052 encodes a PBEF with identity to NadV. SEQ ID NO:21 is a nucleic acid from Suberites domucula (sponge; GenBank Accession No. Y18901) in which codons 317 to 1735 encodes a PBEF with identity to NadV.

Further still, the above methods and vaccines further includes using nucleic acids encoding a protein with identity to the NadV proteins provided herein isolated from eukaryotes such as humans, aquatic organisms such as carp and sponges, and mammals and prokaryotes such as V-factor independent bacteria selected from the group consisting of Actinobacillus actinomycetemcomitans, Actinobacillus lignieresii, Actinobacillus pleuropneumoniae, Actinobacillus suis, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Pasteurella haemolytica, and Pasteurella multocida, and mutants thereof.

The following examples are intended to promote a further understanding of the present invention.

### EXAMPLE 1

This example shows the cloning and sequence analysis of the nadV gene from a V-factor independent strain of H. ducreyi. As shown in the example, a recombinant V-factor in dependent Actinobacillus pleuropneumoniae (APP) was constructed by transforming the NadV gene into a V-factor dependent strain of APP. The example also shows that homologues of the NadV appears to be widely distributed among both prokaryotic and eukaryotic organisms thus indicating the present invention can be used to construct a wide variety of recombinant microorganisms.

### Materials and Methods

Bacterial strains and growth conditions. E. coli XL 1-Blue MRF′ (commercially available from Stratagene, La Jolla, Calif.) was used for propagation of the plasmid pUC18 (commercially available from Gibco-BRL, Rockville, Maryland) and the E. coli-A. pleuropneumoniae shuttle vector, pGZRS18 (West et al., Gene. 160(1): 81-6 (1995)) as well as derivatives of these plasmids. E. coli strains were grown on Luria-Bertani (LB) medium supplemented with ampicillin (100 μg/ml) for plasmid selection. A. pleuropneumoniae (APP; ATCC 27088) and H. influenzae KW20 Rd-(Bricker et al., Proc. Natl. Acad. Sci. USA. 80(9): 2681-5. (1983)) strains were grown at 37° C. under a 5% CO₂ atmosphere on brain heart infusion (BHI) broth or agar (Difco Laboratories, Detroit, Mich.) supplemented with V-factor (NAD) and X-factor (hemin), both at 10 μg/ml and ampicillin at 50 μg/ml as needed. NAD was omitted when selecting for V-factor independent transformants. H. ducreyi ATCC 27722 was grown on chocolate agar (BHI agar base plus 5% boiled she blood plus 1% IsoVitalex) at 35° C. in a candle jar.

The defined medium used to grow APP and H. influenzae was a modification of the recipe developed by Herriott for H. influenzae (Herriott et al., J. Bacteriol. 101(2): 517-24 (:970)), with 10 mM glucose added and the amino acid stock solution from the Neisseria defined medium developed by Morse and Bartenstein (Can. J. Microbiol. 26(1): 13-20 (1980)) substituted for Herriott's amino acid solution. This medium was supplemented with 10 μg/ml hemin, and with 10 μg/ml NAD or nicotinamide (Sigma Chemical Co., St. Louis, Mo.) as needed to determine specific nutritional requirements.

DNA manipulations. Restriction enzymes, calf intestinal phosphatase, and DNA ligase were purchased from Boehringer Mannheim Biochemicals (Indianapolis, Ind.) and used according to the manufacturer's instructions. DNA fragments for subcloning were purified from agarose gels by excising the bands and isolating the DNA with QIAEX beads (Qiagen Inc., Valencia, Calif.) Plasmid DNA was isolated from E. coli, A. pleuropneumoniae, H. ducreyi and H. influenzae using the QIAPREP-spin plasmid purification kit (Qiagen). E. coli was transformed with plasmids using the method of Hanahan (J. Mol. Biol. 166(4): 557-80 (1983)). Plasmids were transformed into H. influenzae using methods described by Herriott (J. Bacteriol. 101(2): 517-24 (1970)). Plasmids were introduced into APP by electroporation as previously described (Fuller et al., Infect. Immun. 64(11): 4659-64 (1996)).

DNA sequencing. Templates for DNA sequence analysis were constructed by subcloning fragments generated from defined restriction sites within pNAD1 into pUC18. Remaining gaps in the sequence were filled using synthetic oligonucleotide primers made at the Macromolecular Structural Facility at Michigan State University as primers for sequencing. DNA sequencing was performed using an ABI100 Model 377 automated sequencer (Applied Biosystem, Foster City, Calif.). Sequence analysis was performed using the web-based Genetics Computer Group package of programs (Genetics Computer Group. Program Manual for the Wisconsin Package, 10^{th} Ed. Genetics Computer Group, Madison, Wis., (1999)). Database searches were performed using the BLAST program provided by the National Center for Biotechnology Information (NCBI) (www.ncbi.nlm.nih.gov). Partially sequenced genomes were accessed and searched either from the NCBI genome database, or from individual databases listed in and linked to The Institute for Genome Research website at http://WWW.tigr.org. The sequences reported for the DNA encoding NadV have been submitted to GenBank and given the accession number AF27384

PCR product subcloning. The ORF predicted to encode the nadV gene was amplified using synthetic primers MM 199 (5′-GCC TGC AGA AAA ATC TTT TGA ATT ATA TAA ACA AC-3′) (SEQ ID NO:11) and MM 191 (5′-GCG TAT TAA CTG CAG ATA TCA TAG CGT AGT GCG-3′) (SEQ ID NO:12), which were designed to introduce unique PstI restriction enzyme sites at either end of the ORF encoding the NadV. The amplification product was digested with PstI and ligated into pUC18 to produce pCNAD9. The insert was then cloned into the E. coli-A. pleuropneumoniae shuttle vector pGZRS18 in both forward and reverse directions to produce pGZNAD9 and pGZNAD10,respectively. Plasmids pGZNAD9 and pGZNAD10 were transformed into APP to produce recombinant APP.

Enzyme assay. The assay for synthesis, of NAD from nicotinamide was adapted from that of Kasarov and Moat (Biochim. Biophys. Acta320(2): 372-8 (1973)). A. pleuropneumoniae serotype 1A containing either pGZNAD9 or pGZRS18 were grown overnight at 37° C. in BHI broth containing 10 μg/ml NAD and 50 μg/ml ampicillin. Cells were harvested by centrifugation, washed in sterile 0.9% saline, suspended in 0.1% of the original culture volume, and disrupted by son cation on ice. Cell debris was pelleted by centrifugation. Cell-free supernatant fractions were combined in a reaction mix that contained 1 ml supernatant fraction, 80 mM potassium phosphate buffer (pH 7.4), 16 MM MgCl₂, 1 mM ATP, 5 mM phosphoribosyl pyrophosphate (PRPP, Sigma), and 2 mM nicotinamide, and the mix incubated at 37° C. in a water bath shaker. At designated time points, 250 μl aliquots were removed and combined with 250 μl saline and 500 μl methanol to stop the reaction.

Analysis of products was performed by HPLC using a Hewlett-Packard model 1050 system with an Alltech LiChrosorb RP-18 column (10 μm particle size, 250×4 mm) equipped with guard column (LiChrosorb RP-18, 5 μm particle size, EM Separations, Wakefield, Rhode Island). The mobile phase consisted of two elements, with an elution gradient as described in Michelli and Sestini, Meth. Enzymol. 280: 211-221 (1997). Eluant A was 8 mM tetrabutylammonium bromide (HPLC grades Sigma) in 0.1 M KH₂PO₄₁ pH 6.0. Eluant B was 70% eluant A and 30% ethanol. Absorbance was measured at 254 nm.

In assays containing radioactive substrate, assay conditions were identical except 350 μM carbonyl-¹⁴C nicotinamide (American Radiolabeled Chemicals, Inc., St. Louis, Mo.) was added in place of the 2 mM nicotinamide. To assay for radioactive incorporation, column fractions were collected into 10 ml Safety Solve scintillation cocktail and samples counted in a Beckman LS 6500 scintillation counter.

### Results

Isolation of the NAD independence plasmid from H. ducreyi. H. ducreyi ATCC 27722 had previously been shown to contain a 5.25 kb plasmid which possessed the ability to confer NAD independence to H. influenzae (Windsor et al., J. Genl. Microbial. 137(Pt 10): 2415-21 (1991)). That finding was corroborated herein by purifying the plasmid DNA from H. ducreyi 27722, using the plasmid DNA to transform an NAD-dependent strain of H. influenzae, and selecting for the ability of transformants to grow on comp ex media in the absence of NAD. One of the NAD-independent colonies recovered was selected, and its plasmid Content was analyzed. The transformant contained a single plasmid of about 5.2 kb. The plasmid was used to re-transform H. influenzae and NAD-independent colonies were again recovered, which carried the 5.2 kb plasmid, confirming that the NAD-independence phenotype was conferred by the 5.2 kb plasmid. Thus, the H. ducreyi plasmid was designated pNAD1.

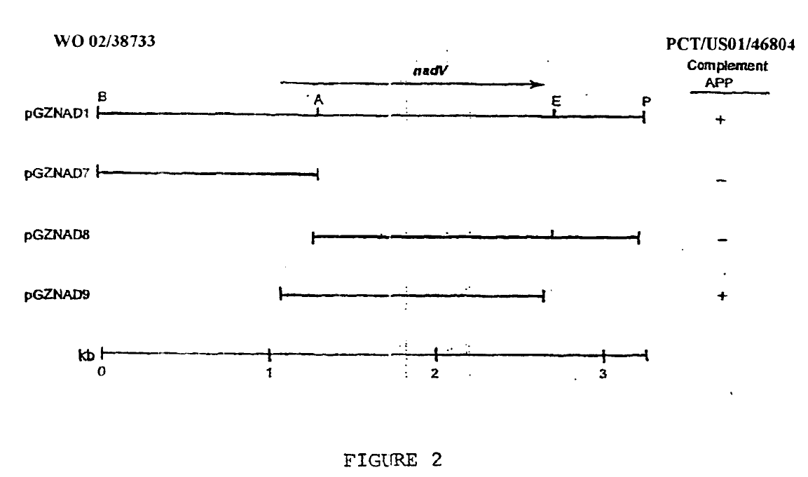

Localization of the NAD independence locus on pNAD1. Plasmid pNAD1 was digested with a variety of restriction enzymes, and an initial restriction map of this plasmid was used to direct the subcloning of fragments of pNAD1 into the cloning vector pUC18. The largest, a 3.3 kb BamHI/PstI fragment, was subcloned into the E. coli-A. pleuropneumoniae shuttle vector pGZRS18 to determine whether the fragment contained the NAD-independence locus. This subclone, pGZNAD1, was electroporated into A. pleuropneumoniae, and transformants plated onto BHI agar lacking NAD. Six of the APP recombinant colonies recovered were found to contain a plasmid of identical restriction pattern to pGZNAD1. This revealed that the gene for NAD independence was functional in A. pleuropneumoniae and was located on the 3.3 kb BamHI/PstI fragment of pNAD1 (FIG. 2).

Sequence analysis of pNAD1. The complete insert of pGZNAD1 was sequenced. The insert was 3307 bp in length and had a G+C content of 34%. The high A+T content of the DNA resulted in a high frequency of stop codons in all three reading frames. One large ORF of 1,482 bp in length was predicted to encode a protein of 494 amino acids with a molecular weight of 55,619 Daltons. There was an AvaI site located 230 bp into the open reading frame. Deletions made from AvaI site in pGZNAD1 resulted in a loss of ability to complement the NAD dependence of A. pleuropneumoniae (FIG. 2). Based on the above genetic evidence linking the ORF to the ability to confer V-factor independence to A. pleuropneumoniae and H. influenzae, the gene encoded by the ORF was designated NadV.

To confirm that the NadV conferred NAD independence, synthetic primers were used to PCR amplify the region containing the nadV and 75 bp upstream of the start codon, and the 1588 bp PCR product was cloned into pGZRS18 in both orientations to form pGZNAD9 (FIG. 2) and pGZNAD10. APP recombinants containing plasmid pGZNAD9 were NAD-independent, but APP recombinants containing plasmid pGZNAD10 were not, suggesting that the NadV gene was expressed from a promoter in the plasmid rather than from its native promoter.

The complete nucleotide sequence (SEQ ID NO:1) and predicted amino acid sequence (SEQ ID NO:2) of NadV have been submitted to GenBank and given the accession number AF273842. A putative ribosome binding site (RBS) was found upstream of the start codon of NadV. No significant inverted repeat sequences characteristic of transcriptional terminators ere found downstream of the stop codon of nadV.

The amino acid sequence of NadV was analyzed for the presence of functionally conserved motifs. The encoded NadV protein did not contain a hydrophobic, N-terminal leader sequence characteristic of secreted proteins, nor did it contain any long stretches of internal hydrophobic resides which could serve as membrane anchors. When compared to a protein motifs database (Genetics Computer Group. Program Manual for the Wisconsin Package, 10^{th} E Genetics Computer Group, Madison, Wis., (1999)), no significant matches were found to conserved regions of previously identified protein families.

Homologues of the nadV gene in other organisms. The NadV amino acid sequence was used to search sequence databases. The search identified one protein with a unrelated function, and seven matches to proteins of unknown function from partially or completely sequenced microbial genomes. The protein with the unrelated function was the human pre-B-cell colony enhancing factor (PBEF) protein (SEQ ID NO:7) (Samal et al., Mol. Cell. Biol. 14(2): 1431-7 (1994)). The homologues discovered in the bacterial genome databases were found in a diverse array of species, including the cyanobacterium Synechocystis (SEQ ID NO:6); the radiation-resistant organism Deinococcus radiodurans (SEQ ID NO:5); two Mycoplasma species, M. genitalium (SEQ ID NO: and M. pneumoniae (SEQ ID NO:9); the Gram negative aquatic and soil organism Shewanella putrefaciens (SEQ ID NO:10); and two NAD-independent members of the Pasteurellaceae, Pasteurella multocida (SEQ ID NO:4) and Actinobacillus actinomycetemcomitans (SEQ ID NO:3). Pair-wise comparisons of these sequenes revealed that NadV had the highest similarity to the homologue from S. putrefaciens, and that these were more closely related to, the Mycoplasma homologies than to the remaining sequences. All nine sequences were aligned (FIG. 3) and numerous regions were found which contained clusters of highly conserved amino acid residues. Also conspicuous were regions where the sequences or sequence gaps from A. actinomycetemecomitans, P. multocida, D. radiodurans, Synechocystis and human PBEF were identical but different from sequences from M. genitalium, M. pneumoniae, S. putrefaciens, and the H. ducreyi NadV. This clustering is indicative of two broad families existing among the homologues of NadV.

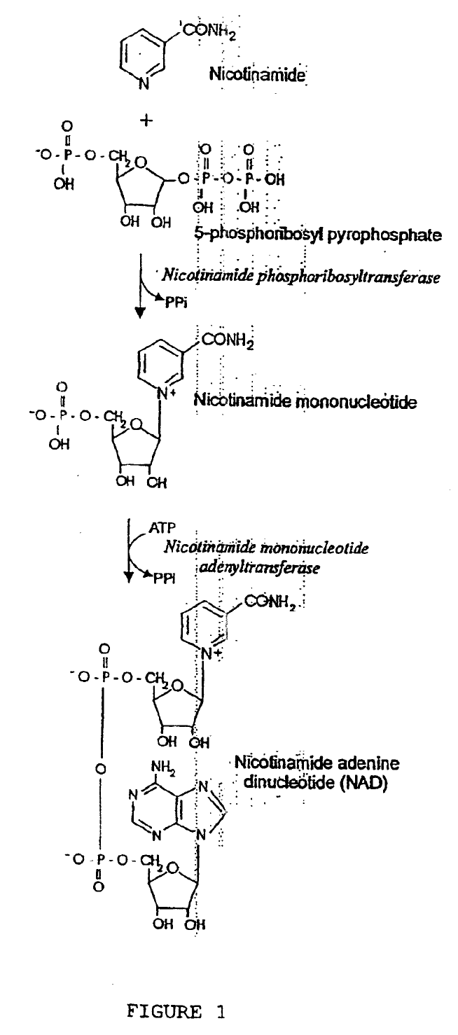

Functional analyses of the NAD independence locus. Previous studies have shown that NAD-independent members of the family Pasteurellaceae differ from the NAD-dependent members solely in their ability to utilize the NAD precursor nicotinamide as V-factor (Niven and O'Reilly, Intl. J. Syst. Bacteriol. 40(1): 1-4 (1990); O'Reilly and Niven, Can. J. Microbiol. 32(9): 733-7 (1986)) To determine whether nadV was responsible for this difference, APP recombinants containing pGZNAD1, pGZNAD9, or the pGZRS18 vector were plated onto defined media lacking V-factor, and onto defined media containing either NAD or nicotinamide. All three strains failed to grow in the absence of supplement and grew in the presence of NAD, but only the strains containing the cloned nadV gene could grow in the presence of nicotinamide. This indicated that the presence of the NadV gene allowed the A. pleuropneumoniae to utilize nicotinamide as a precursor for NAD biosynthesis as diagramed in FIG. 1, and suggests that the enzyme encoded by this gene is a novel nicotinamide phosphoribosyltransferase (NAm-PRTase).

Assay for NAm-PRTase activity. Crude cell extracts were prepared from APP recombinants containing either pGZNAD9 or the pGZRS18 vector and assayed for the abillity to synthesize NMN and NAD from nicotinamide plus PRPP. As shown in Table 1, NAm-PRTase assays performed with extracts of A. pleuropneumoniae containing pGZNAD9 showed a decrease in NAm and a concomitant increase in NAD as well as a slight, but consistent, increase in the levels of NMN. APP recombinants containing the pGZRS18 vector alone did not show an equivalent increase in NAD or decrease in nicotinamide, nor was this pattern seen when assays with APP recombinants containing pGZNAD9 were performed without PRPP in the reaction mix.

TABLE 1
Synthesis of NAD from nicotinamide and PRPP
by extracts of A. pleuropneumoniae containing nadV^{a,b}

| | NAm | NMN | NAD |
|---|---|---|---|
| Time | (μmoles) | (μmoles) | (μmoles) |
| 0 | 360 | 25 | 25 |
| 30 | 220 | 40 | 123 |

^{a}Reaction mixture contained cell extract; 80 mM potassium phosphate buffer, pH 7.4; 16 mM MgCl₂; 1 mM ATP; 5 mM PRPP; and 2 mM nicotinamide, and the reaction mixture was incubated for 30 minutes at 37° C.
^{b}Data presented is from a representative experiment. Trends were identical in all experiments.

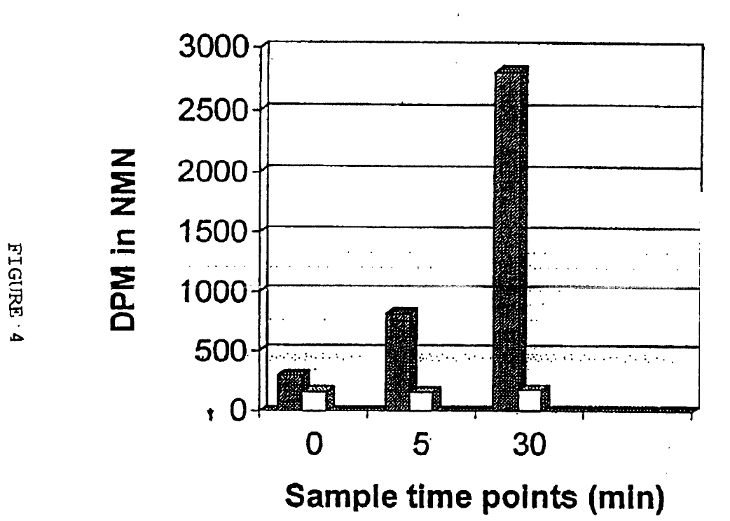

To confirm that NMN is indeed an intermediate in the biosynthesis of NAD from nicotinamide as catalyzed by the NadV gene product, ¹⁴C-nicotinamide was used as substrate in the same assay system. As shown in FIG. 4, ¹⁴C-label was incorporated into NMN by cell extracts from APP recombinants containing pGZNAD9, but not in control reactions with extracts from APP recombinants containing pGZRS18.

### Discussion

This example shows the cloning, sequence analysis, and characterizaion of a plasmid-encoded gene, NadV, from H. ducreyi which confers V-factor independence to several species of V-factor dependent Pasteurellaceae. A 5.25 Kb plasmid from H. ducreyi 27722 was previously described by Windsor et al. (Med. Microbiol. Lett. 2: 159-167 (993)), and shown to confer V-factor independence to H. influenzae and H. parainfluenzae. Similar plasmids have been described in V-factor independent strains of H. parainfluenzae (Windsor et al., Intl. J. Syst. Bacteriol. 43(4): 799-804(1993)) and H. paragallinarum (Bragg et al., J. Vet. Res. 60(2): 147-52 (1993)). However, the plasmid gene or genes responsible for conferring V-factor independence were not identified.

As shown herein, a single gene on the plasmid, nadV, was discovered to be responsible for the V-factor independent phenotype. Further, as shown herein, subclones consisting of DNA from the 5.2 kb plasmid inserted into E. coli-A. pleuropneumoniae shuttle vectors and transformed into a different member of the family Pasteurellaceae, A. pleuropneumoniae (APP), produced APP recombinants which were V-factor independent. Therefore, the ability of the NadV to confer V-factor independence to V-factor dependent bacteria is not restricted to V-factor dependent strains of H. ducreyi but includes other members of the Pasteurellaceae family and is expected to include bacteria for other families which have a similar biosynthesis pathway for synthesizing NAD.

Members of the family Pasteurellaceae are incapable of either de novo synthesis of NAD via quinolinic acid or of recycling of pyridine nucleotides via nicotinic acid (Cynamon et al., J. Gen. Microbiol. 134(Pt. 10): 2789-99 (1988); Foster et al., Microbiol. Rev. 44(1): 83-105 (1980); Niven and O'Reilly, Intl. J. Syst. Bacteriol. 40(1): 1-4 (1990)), which leads to their requirement for an exogenous source of pyridine nucleotide, or V-factor. V-factor dependence in the Pasteurellaceae family has been defined as the requirement for either NAD, NMN or NR for growth on complex media (Niven and O'Reilly, Intl. J. Syst. Bacteriol. 40(1): 1-4 (1990). Using this definition, species such as H. influenzae, H. parainfluenzae, H. parasuis, and A. pleuroneumoniae are V-factor dependent, while P. multocida, P. haemolytica, H. haemoglobulinophilus, and A. actinomycetemcomitans are not V-factor dependent. However, all of the members of the Pasteurellaceae family require a pyridine nucleotide when grown on chemically defined media (Niven and O'Reilly, Intl. J. Syst. Bacteriol. 40(1): 1-4 (1990)). In this case, the difference is that the V-factor independent strains can utilize NAm as the pyridine nucleotide, as well as utilizing NAD, NMN, and NR, but the V-factor dependent strains can not utilize NAm. This distinction between V-factor dependent and V-factor independent strains based on growth on complex media is somewhat artificial, since most complex media contain significant amounts of NAm (Niven and Levesque, Intl. J. Syst. Bacteriol. 38(3): 319-320 (1988); Niven and O'Reilly, Intl. J. Syst. Bacteriol. 40(1): 1-4 (1990)).

Niven and O'Reilly (Intl. J. Syst. Bacteriol. 40(1): 1-4 (1990)) proposed that the distinction between V-factor independent and dependent strains in the family Pasteurellaceae may reflect the presence or absence of a single enzyme, NAm phosphoribosyltransferase, to convert NA to NMN. The results shown herein are consistent with the proposal. As shown herein, the APP recombinants expressing NadV had the ability to grow on a complex medium without added V-factor and that the presence of the NadV gene also enabled the APP recombinants to grow on a chemically defined medium containing NAm but lacking an exogenous source of pyridine nucleotide. Also shown was that NAD could be synthesized from NAm and PRPP via NMN in the APP recombinant, which supports the conclusion that NadV encodes an NAm phosphoribosyl-transferase. In addition, in an analysis of currently available genomic databases, homologues of NadV were found in P. multocida and A. actinomycetemcomitans, two V-factor independent species, but not in H. influenzae, which is V-factor dependent.

As shown in FIG. 3, homologues of NadV were found from a variety of highly diverse bacterial species, including two mycoplasmas; a cyanobacterium, a Gram negative aquatic and soil bacterium, and a Gram positive radiation-resistant coccus. The H. ducreyi, nags gene was more closely related to the homologues found in Shewanella and in Mycoplasma species than to either the P. multocida or A. actinomycetemcomitans homologues. This likely indicates that horizontal transfer of this gene has occurred. The NadV gene is located on a plasmid in H. ducreyi, but in the chromosome of the other bacterial species. One possibility is that the nadV gene moved into H. ducreyi from M. genitalium. A similar horizontal transfer has been proposed as the source of the term gene found in most urogenital pathogens of humans (Roberts et al., Antimicrob. Agents Chemother. 30(5): 810-2 (1986)). We did not find NadV homologues in a wide variety of other species, including members of the Enterobacteriaceae and Bacillaceae, known to either synthesize NAD de novo or to possess pyridine salvage pathways.

The only homologue of NadV with a proposed function to date is human pre-B-cell colony enhancing factor (PBEF) (Samal et al., Mol. Cell. Biol. 14 (2): 1431-7 (1994)). The human PBEF gene was transcribed mainly in human bone marrow, liver, and muscle cells as well as in activated human lymphocytes. It was proposed to encode a novel cytokine-like molecule that enhanced the effect of stem cell factor and interleukin-7 on B-cell development, but this has not been studied further. The function of NadV in the biosynthesis of NAD should provide an important clue as to the role of PBEF in mammalian species.

The sequences identified in microbial genome sequencing projects, which are shown herein to have identity to the NadV of the present invention, were designated as homologues of PBEF. For M. genitalium, the similarity led to the hypothesis that the gene sequence encoding the homologues of PBEF could be linked to pathogenicity via a potential immune regulatory function (Ouzounis et al., Mol. Microbiol. 20(4): 898-900 (1996)). However, the discovery that the PBEF homologues have identity to the NadV of the present invention provides a more plausible explanation for the role of the gene product in bacterial metabolism and will be useful in future microbial genome analyses as an indicator of the presence of an alternative NAD biosynthetic pathway.

The requirement for V-factor is a key taxonomic criterion for identification of members of the Pasteurellaceae. The result shown herein indicate that the inability to utilize NAm to fulfill this requirement is due to the absence of a single gene, the nadV gene of the present invention. Further shown herein is that while two V-factor independent species, P. multocida and A. actinomycetemcomitans, possess chromosomal copies of a homologue of the nadV gene, H. influenzae, the only V-factor dependent species for which a complete genome sequence is available, does not possess the nadV gene. The location of the H. ducreyi NadV gene on a plasmid and its apparent mobility into other V-factor dependent species of haemophili suggests that the use of NAD requirements for identification of individual members of the Pasteurellaceae may prove problematic in future. However, for the present, NAD-independence is not widespread in H. ducreyi, H. paragallinarum, H. parainfluenzae, or A. pleurapneumoniae; therefore, it seems feasible to continue to use NAD dependence as a taxonomic criterion with the caveat that NAD-independent strains of these species do exist.

### EXAMPLE 2

This example shows construction of a recombinant Actinobacillus pleuropneumoniae (APP) wherein NadV selection is used to isolate the recombinant APP.

As shown in Example 1, expression of the NadV gene was dependent upon the orientation of the gene in the shuttle vector. The ability of the recombinant APP Containing the NadV to grow in the absence of exogenous NAD was seen only when NadV was cloned in the forward direction (pGZNAD9), and not in the reverse direction (pQZNAD10). This suggested hat expression of NadV was from a promoter in the pGZRS18 vector rather than from its own promoter. Therefore, to construct an NadV gene expression cassette, a promoter that functions in APP was inserted upstream of the nadV to allow expression of the NadV gene independent of its orientation in a plasmid.

APP strains were cultured at 37° C. in either brain heart infusion (BHI),heart infusion (HI), or tryptic soy agar (TSA) (Difco Laboratories, Detroit, Mich.) containing 10 μg/m NAD (V factor) (Sigma Chemical Company, St. Louis, Mo.) when needed. Isoleucine and valine (Sigma) were added to a final concentration up to 200 μg/ml when needed. E. coli strains were cultured in Luria-Bertani medium. Ampicillin was added at 10 μg/ml to 50 μg/ml for plasmid selection in E. coli trains. For APP strains, 10 μg/ml NAD was added as required, except for selection after transformation which were performed without addition of NAD.

DNA modifying enzymes were supplied by various commercial sources and used according to the manufacturer's specifications. Plasmid DNA preparations, agarose gel electrophoresis, and E. coli transformation were all performed by conventional methods (Sambrook et al. (Eds.), In: Molecular Cloning: A Laboratory Manual, 2nd. ed. Cold Spring Harbor Press, Cold Spring Harbor, N.Y. (1989)).

The pUCK4K plasmid (Taylor and Rose, Nucl. Acids Res. 16 (1), 358 (1988)) contains a kanamycin expression cassette that is constitutively expressed in APP, independent of its orientation in the expression vector. The pUC4K plasmid is commercially available from Pharmacia Biotech, Piscataway, N.J. The promoter from the pUC4K kanamycin resistance cassette was inserted in front of the nadV gene to produce an NadV gene expression cassette, which allowed expression of the nadV independent of its orientation in the expression vector, as follows.

The kanamycin cassette in pUC4K was flanked by polynucleotides comprising nested restriction enzyme sites which are useful in cloning. The promoter region of the kanamycin gene was PCR amplified using PCR primers that flanked the promoter region. PCR primer 1 was located upstream of the nested restriction enzyme sites and primer 2 was centered over the start codon of the protein encoded by the kanamycin gene. PCR primer 2 also contained a NcoI restriction enzyme site.

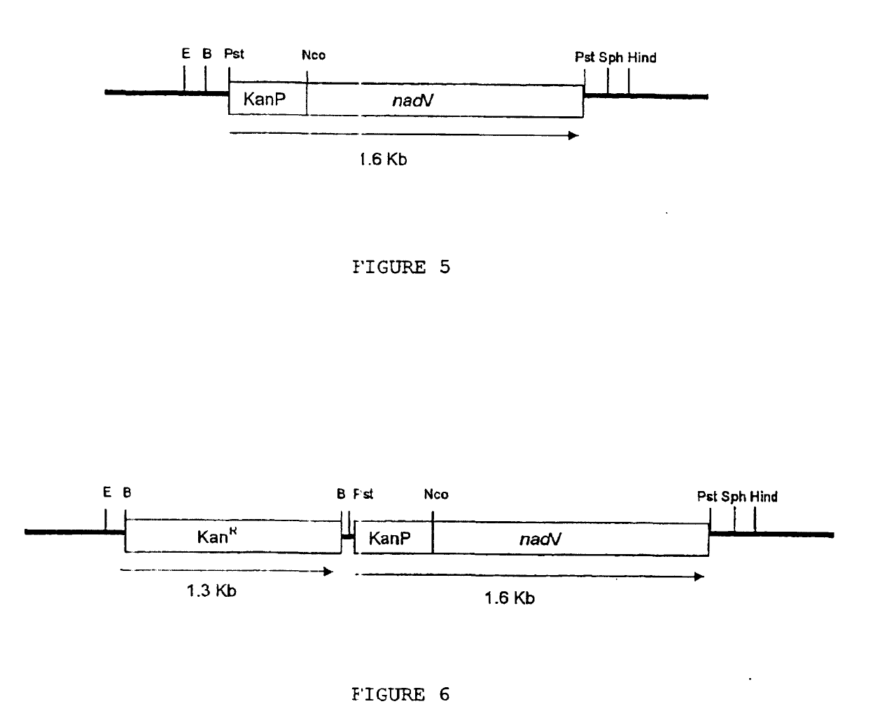

The PCR product containing the kanamycin promoter was digested with PstI (the site of which was located in the nested restriction enzyme sites upstream of the kanamycin promoter) and NcoI. The NadV gene was PCR amplified using PCR primers designed to incorporate a NcoI site at the ATG star codon of the gene and to retain a PstI site that was immediately downstream of the gene's stop codon. The nadV PCR product was digested with NcoI and PstI. A pUC18 plasmid for receiving the digested PCR products was digested with PstI and a three-way ligation consisting of the kanamycin promoter region, the nadV gene, and the pUC18 plasmid was performed to produce plasmid pC18KnadV (FIG. 5) which contained the NadV gene expression cassette with the kanamycin promoter region operably linked to the nadV gene.

Next, the NadV gene expression cassette from pC18KnadV was cloned into both the pGZRS18 and the pGZRS19 E. coli-A. pleuropneumoniae shuttle vectors to produce pGZ18KnadV and GZ19KnadV, respectively. Shuttle vector pGZRS19 is described by West et al. (Gene 160: 81-86 (1995) and is obtainable by digesting plasmid pTF76 with HindIII to remove the ribGBAH operon. Plasmid pTF76 was deposited a the American Type Culture Collection, 10801 University Boulevard, Manassas, Va. as ATCC PTA-2436. These constructs were each electroporated into V-factor dependent A. pleuropneumoniae serotype 1 and plated on BHI agar without exogenous NAD. Both constructs enabled the APP recombinants to grow in the absence of NAD, proving that nadV was properly expressed from the kanamycin promoter independent of its orientaton in the pGZRS vector. These results also reconfirmed that the nadV gene confers NAD independence to PP serotype 1 as shown in Example 1.

### EXAMPLE 3

This example show the construction of an NadV/kanamycin double-selection gene expression cassette wherein the kanamycin gene facilitates construction of the cassette in E. coli and selection of APP recombinants is by either NadV expression or kanamycin resistance.

DNA modifying enzymes were supplied by various commercial sources and used according to the manufacturer's specifications. Plasmid DNA preparations, agarose gel electrophoresis, and E. coli transformation were all performed by conventional methods (Sambrook et al. (Eds.), In: Molecular Cloning: A Laboratory Manual, 2nd. ed. Cold Spring Harbor Press, Cold Spring Harbor, N.Y. (1989))

To make the double-selection gene expression cassette, the kanamycin cassette from pUC4K was cloned into the pC18KnadV plasmid Clones containing this construct confer both kanamycin resistance and NAD independence to recombinant APP. The kanamycin resistance gene was isolated from BamHI digested pUC4K and cloned into BamHI digested pC18KnadV to produce plasmid pC18KanNad (FIG. 6) The orientation of the inserted gene was shown by sequencing pC18KanNad using the pUC forward and reverse primers.

### EXAMPLE 4

This example shows the construction of an attenuated recombinant Actinobacillus pleuropneumoniae (APP) wherein NadV selection is used to isolate an attenuated recombinant wherein a portion of the ilvI gene, which encodes an acetohydroxy acid synthase enzyme, is replaced with the NadV gene.

APP strains were cultured at 37° C. in either brain heart infusion (BHI), heart infusion (HI), or tryptic soy agar (TSA) (Difco Laboratories, Detroit, Mich.) containing 10 μg/ml NAD (V factor) (Sigma Chemical Company, St. Louis, Mo.) when needed. Isoleucine and valine (Sigma) were added to a final concentration of up to 200 μg/ml when needed for growing recombinant APP. E. coli strains were cultured in Luria-Bertani medium. Kanamycin was added at 100 μg/ml for plasmid selection in E. coli strains. For APP strains, 10 μg/ml NAD was added as required, except for selection after transformations which were performed without addition of NAD.

The NadV/kanamycin double-selection gene expression cassette was used to construct an ilvI knock-out cassette for making a recombinant APP wherein a portion of the ilvI gene in the APP genome was replaced with the NadV/kanamycin double-selection gene expression cassette. The APP ilvI gene was identified and shown to be homologous to similar genes in a variety of other bacterial species (Fuller et al., Microb. Pathol. 27(5): 311-327 (1999)). The ilvI gene encodes an acetohydroxy acid synthase enzyme involved in the biosynthesis of isoleucine and valine. Disruption of the ilvI gene in APP results in a non-lethal mutation, provided that exogenous isoleucine, leucine, and valine (ILV) are supplied to the APP.

Construction of an ilvI knockout cassette for making an attenuated APP by homologous recombinant was made as follows. First, a deletion-disruption vector comprising an ilvI gene cassette with the 5′ and 3′ ends of the ilvI gene in a plasmid vector was made. In the ilvI gene cassette, the 0.3 Kb internal coding region of the APP ilvI gene was deleted and replaced with the NadV/kanamycin double-selection gene expression cassette.

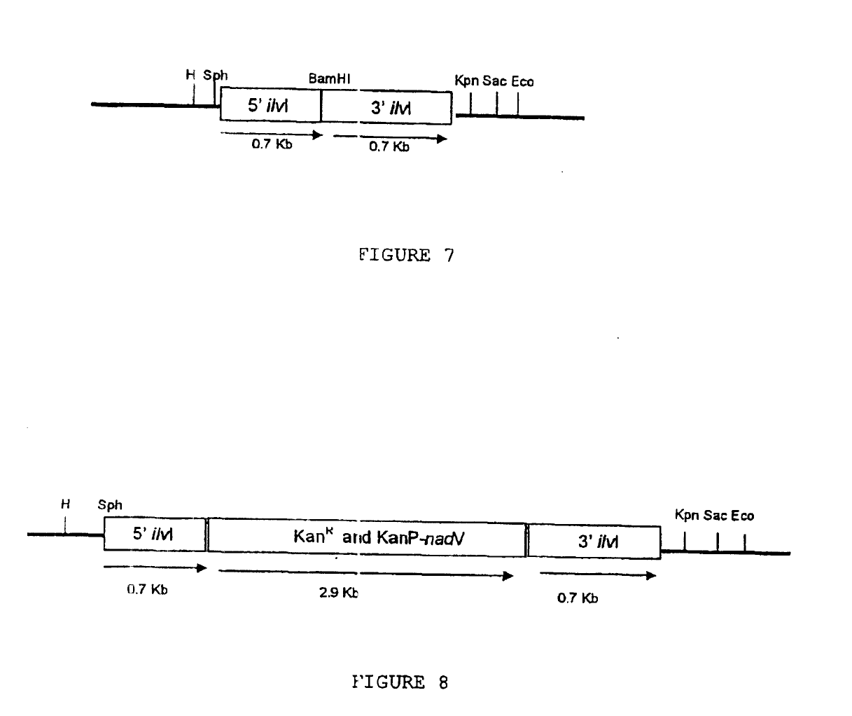

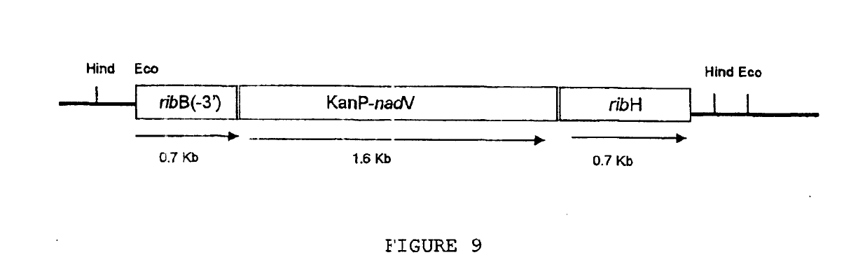

To construct the ilvI knockout cassette, the 3′ end of the ilvI was PCR amplified from A. pleuropneumoniae genomic DNA using Pfu polymerase and cloned into pUC18 digested with SmaI to produce pilvI3′ Next, the 5′ end of the ilvI was PCR amplified with PCR primers designed to incorporate BamHI and SphI restriction enzyme sites into the PCR product. Both the ilvI 5′ PCR product and pilvI3′ were digested with BamHI and SphI and the digested 5′ product and pilvI3′ were ligated together to produce plasmid pilvI5′3′ (FIG. 7). Plasmid pilvI5′3′ contained the 0.7 Kb of the 5′ end of the ilvI and 0.7 Kb of the 3′ end of the ilvI separated by a BamHI site, but did not contain the internal 0.3 Kb of the ilvI.

Plasmid pilvI5′3′ was digested with BamHI and the single-stranded ends were made blunt using Klenow polymerase. The NadV/kanamycin double-selection gene expression cassette was PCR amplified from puC18KanNad with Pfu polymerase using th pUC18 forward and reverse primers. Pfu polymerase yields blunt ends on PCR products. The NadV/kanamycin double-selection gene expression cassette was ligated into the blunt-ended BamHI site of pilvI5′3′ to produce pC18ilvKanNad (FIG. 8.)

Construction of an ilvI knockout APP recombinant (ilvI-APP recombinant) by homologous recombination. pC18ilvKanNad was isolated from E. coli XL1-Blue mrF and was introduced into competent A. pleuropneumoniae serotype 1 cells by electroporation. Transformants were allowed to recover for 4 hours in the presence of NAD and the amino acids, isoleucine, leucine, and valine (ILV). Transformants were plated on BHI with isoleucine, leucine and valine but without NAD to select V-factor independent APP recombinants. After 48 hours, transformant colonies were transferred to BHI with isoleucine, leucine, and valine containing kanamycin (100 μg/ml). Over 96% of the colonies that grew on the BHI also grew on BHI with kanamycin.

Colonies that were V-factor independent and kanamycin resistant were su cultured onto BHI lacking either NAD, ILV amino acids, or both. Four colonies of recombinant APP were selected which could not grow in the absence of exogenous isoleucine and valine. Genomic DNA was prepared from the recombinant APP in those colonies as well as from appropriate controls and the DNA was analyzed by Southern blot. The Southern blot demonstrated that all four recombinant APP contained the NadV/kanamycin double-selection gene expression cassette inserted into the APP's ilvI gene. However, because the recombinant APP also contained the pUC18 vector backbone inserted into the APP's ilvI gene, the APP recombinants were produced from single crossover recombination events. No double crossovers nor wild type colonies were identified.

The results demonstrated that the NadV gene can be expressed efficiently in single copy in the bacterial chromosome, and that the NAD independence phenotype conferred by the presence of the nadV gene can be used to select recombinant APP.

### EXAMPLE 5

This Example show the construction and analysis of a stable attenuated recombinant of A. pleuropneumoniae (APP) using the NadV/kanamycin double-selection gene expression cassette and the NadV gene expression cassette of Example 4.

The single crossover ilvI-APP recombinant described in Example 4, in which the entire pC18KanNad plasmid was inserted into the ilvI gene, in some cases may be too unstable to maintain the ilvI-phenotype, particularly when the recombinant APP is introduced into pigs. Therefore, this example shows the construction of a stable double crossover ilvI-APP recombinant in which the central portion of the ilvI gene is replaced with either the NadV/kanamycin double-selection gene expression cassette from pC18KanNad or the NadV gene expression cassette from pC18 KnadV.

To construct an APP recombinant with the NadV/kanamycin double-selection gene expression cassette, the above 7.1 Kb pC18ilvKanNad plasmid is used. The plasmid is linearized using Sph1, treated with calf alkaline phosphatase to prevent recircularization of the plasmid, and electroporated into APP serotype 1 (APP-1). Transformants are selected on BHI+ILV (isoleucine, leucine, and valine) agar with no NAD added, which selects for expression of the NadV gene and NAD independence. Transformants are further screened for lack of growth in the absence of exogenous isoleucine, leucine, and valine (ILV). ILV requiring, NAD-independent transformants (ilvI-APP recombinants) are analyzed by Southern blots. Genomic DNA is prepared from each transformant of interest, digested with the restriction enzyme ClaI, and separated by agarose gel electrophoresis. DNA fragments are transferred to nitrocellulose membranes, an the resulting blot probed for bands homologous to (1) the intact ilvI gene; (2) the 300 bp ilvI internal fragment deleted from the ilvI gene in pC18ilvKanNad; (3) the intact NadV gene from pC18KnadV; and (4) the kan gene from pUC4K. Predicted sizes of the DNA fragments resulting from either single or double crossover events are shown in Table 2.

TABLE 2

| | | Double | Single |
|---|---|---|---|
| Probe | WT APP-1 | crossover | crossover |
| ilvI | 5.5 kb | 8.2 kb | 12.6 kb |
| 300 bp ilvI | 5.5 kb | — | 12.6 kb |
| nadV | — | 8.2 kb | 12.6 kb |
| kan | — | 8.2 kb | 12.6 kb |

The Southern blot data is used to confirm the correct construction of the double-crossover ilvI-APP recombinant. Stability of the ilvI- and NAD independent phenotypes during growth of the strain under non-selective conditions is also confirmed by passage through pigs or the like.

Similar methods re used to construct a recombinant APP with the nadV gene expression cassette. A plasmid for homologous recombination is constructed which contains 0.7 kb of the 5′ end of ilvI, the 1.6 kb NadV expression cassette with the KanP promoter, and 0.7 kb of the 3′ end of ilvI. Once constructed, the 5.7 Kb plasmid is used for knockout construction as described above for pC18ilvKanNad. In this case, predicted sizes of the DNA fragments resulting from either single or double crossover events are shown in Table 3.

TABLE 3

| | | Double | Single |
|---|---|---|---|
| Probe | WT APP-1 | crossover | crossover |
| ilvI | 5.5 kb | 6.8 kb | 11.2 kb |
| 390 bp ilvI | 5.5 kb | — | 11.2 kb |
| nadV | — | 6.8 kb | 11.2 kb |
| kan | — | — | — |

Analysis of attenuation of an ilvI-APP recombinant. Attenuation of the ilvI-APP recombinant is evaluated using previously published methods (Fuller et al., Infect. Immunol. 64: 4659-4664 (1996)). Briefly, six groups of 3 pigs each are infected intratracheally as follows: Group (1), 5×10⁶ CFU (1 LD₅₀) of AP225, wild-type (WT) APP serotype 1; Group (2), 5×10⁶ CFU of the ilvI-APP recombinant (equivalent to 1×LD₅₀ for the WT parent strain); Group (3), 2×10⁷ CFU of the ilvI-APP recombinant (equivalent to 4×LD₅₀ for the WT parent strain); Group (4), 1×10⁸ CFU of the ilvI-APP recombinant (equivalent to 20×LD₅₀ for the WT parent strain); Group (5), 5×10⁸ CFU of the ilvI-APP recombinant (equivalent to 100×LD₅₀ for the WT parent strain); and, Group (6), 5×10⁶ CFU of the ilvI-APP recombinant complemented with the intact ilvI gene on a plasmid (equivalent to 1×LD₅₀ for the WT parent strain).

The pigs are monitored every four hours post-infection and scored for clinical signs of pleuropneumonia, including increased respiration rate and temperature; dyspnea; loss of appetite; and change in activity or attitude (depression) (Jolie et al., Vet. Microbiol. 45: 383-391 (199)). Seriously ill animals, as determined by dyspnea and depression scores, are euthanized and necropsied immediately. Survivors are euthanized three days post-infection. All animals are necropsied and the lungs examined macroscopically for signs of A. pleuropneumoniae lesions. Severity and type of lesions are scored using a standard formula. Representative lung samples are collected for histopathology and bacteria culture. Attenuation is assessed as decreased mortality, decreased lung lesions scores, and/or decreased severity of clinical scores in comparison to the group infected with WT APP-1 (Jolie et al., Vet. Microbiol. 45: 38-391 (1995)).

The ilvI-APP recombinant is tested as a live avirulent vaccine against disease cause by A. pleuropneumoniae, using previously established procedures (Fuller et al., Vaccine 18: 2867-2877 (2000)).

### EXAMPLE 6

This example shows the construction of attenuated recombinant A. pleuropneumoniae serotype 1 (APP) by replacing several genes of the riboflavin biosynthesis operon with a gene expression cassette encoding the NadV gene of the present invention.

Disruption of riboflavin synthesis was shown in U.S. Pat. No. 5,925,354 to Fuller et al. to attenuate APP and by using the nadV gene for positive selection, the attenuated recombinant APP is purified from non-recombinant non-attenuated ALP. In general, the method disclosed in U.S. Pat. No. 5,925,354 to Fuller et al. is followed with the exception that the plasmid transformation vectors contain the NadV gene expression cassette instead of the kanamycin gene expression cassette.

APP strains are cultured at 37° C. in either brain heart infusion (BHI), heart infusion (HI), or tryptic soy agar (TSA) (Difco Laboratories, Detroit, Mich.) containing 10 μg/ml MAD (V factor) (Sigma Chemical Company, St. Louis, Mo.) when needed. Riboflavin (Sigma) is added to a final concentration of 200 μg/ml when needed. E. coli strains are cultured in Luria-Bertani medium. Ampicillin is added at 100 μg/ml for plasmid selection in E. coli strains. For APP strains, 10 μg/ml MAD is added as required, except for selection after matings which are performed without addition of NAD. APP strains AP100 and APP225 were deposited at the American Type Culture Collection, 10801 University Boulevard, Manassas, Va. as ATCC 27088 and ATCC PTA-2429, respectively.

DNA modifying enzymes are supplied by various commercial sources and used according to the manufacturer's specification. Genomic DNA is prepared according to the lysis/proteinase K method of the Gene Fusion Manual (Silhavy, In: Experiments with Gene Fusions. Cold Spring Harbor Laboratoy Press, Cold Spring Harbor, N.Y. pp. 137-139(1984)). Plasmid DNA preparations, agarose gel electrophoresis, and E. coli transformation are all performed by conventional methods (Sambrook et al. (Eds.), In: Molecular Cloning: A Laboratory Manual, 2nd. ed. Cold Spring Harbor Press, Cold Spring Harbor, N.Y. (1989)) Plasmids pTF10 and pTF66 were deposited at the American Type Culture Collection, 10801 University Boulevard, Manassas, Va. as ATCC PTA-238 and ATCC PTA-2437, respectively. The riboflavin biosynthesis operon has the nucleotide sequence set forth in SEQ ID NO:18.

To construct riboflavin-requiring auxotrophic mutants of APP using selection based on V-factor independence, a suicide plasmid with part of the riboflavin operon deleted and replaced with a nadV cassette is constructed. A 2.9 kb EcoRI DNA fragment from pTF10 (ATCC PTA-2438) containing the A. pleuropneumoniae ribBAH genes is cloned into the EcoRI site of the conjugative suicide vector pGP704 to create plasmid pTF66 (ATCC PTA p⁴³⁷). Plasmid pTF66 is digested with ClaI and NdeI to excise the 3′ end of ribB and the entire ribA gene. After Klenow treatment of the DNA, the NadV gene expression cassette, which is excised from pC18KnadV with PstI and the ends made blunt, is blunt-end ligated into the rib deletion site to create plasmid pTF66-nadV.

Plasmid pTF66-nadV is transformed into E. coli S17-1 (λpir) and using filter mating targeted mutagenesis, mobilized into AP100 (ATCC 27088) and AP225 (ATCC PTA-2429), which is nalidixic acid resistant, to produce transconjugant colonies which are riboflavin auxotrophs and either V-factor independent or in the case of AP225 conjugates, also resistant to nalidixic acid. Filter mating between E. coli containing plasmid pTF66-nadV and APP is performed according to the protocol of U.S. Pat. No. 5,925,354 to Fuller et al. Briefly, bacterial cultures are grown overnight at 37° C. Equal cell numbers of donor and recipient cultures, as determined by optical density at 520 nm, are added to 5 ml 10 mM MgSO₄ and the bacteria pelleted by centrifugation. The pellet containing the cell mating mixture, resuspended in 100 μl of 10 MM MgSO₄₁ is plated on a sterile filter on BHIV⁺ riboflavin agar and incubated for 3 hr. at 37° C. Cells are then washed from the filter in sterile phosphate buffered saline (pH 7.4), centrifuged, resuspended in 400 μl BHIV broth, and plated in 100 μl aliquots on BHIV containing riboflavin but not NAD.

Colonies that are V-factor independent are selected from filter mating plates and screened for riboflavin auxotrophy by replica plating onto TSAV, observing for inability to grow in the absence of added riboflavin. Transconjugants are replica plated onto TSAV and TSAV+riboflavin to assess the requirement for riboflavin and the stability of the riboflavin auxotrophy. All transconjugants are confirmed as A. pleuropneumoniae by gram stain and colonial morphology. APP riboflavin deletion transconjugants (rib-APP recombinants) which are either V-factor independent and nalidixic acid resistant or V-factor independent are selected for further analysis based on their phenotypes as potential single or double cross-over mutants by Southern blot analysis as taught in U.S. Pat. No. 5,925,354 to Fuller et al. Briefly, chromosomal DNA and plasmid controls are digested with the appropriate restriction enzymes and the DNA fragments were separated on an 0.7% ultrapure agarose gel in TAE buffer. Southern blots are performed as described by Sambrook et al. (Eds.), In: Molecular Cloning: A Laboratory Manual 2nd. ed. Cold Spring Harbor Press, Cold Spring Harbor, N.Y. (1989). DNA probes are labeled with digoxygenin by random priming using the Genius V. 3.0 kit from Boehringer Mannheim Probes include the 5.2 Kb insert from pTF10 contain ng the intact riboflavin operon from AP106 (Rib), the 1.4 Kb ClaI/NdeI fragment deleted from the riboflavin operon in the construction of pTF66-nadV, the NadV gene expression cassette from pC18KnadV and the intact plasmid pGP704 (pGP704). Hybridization is carried out in 50% formamide at 42° C. for 16 hr. Blots are washed twice in 2×SSC/0.1% SDS for 15 minutes at room temperature, then twice in 0.1×SSC/0.1% SDS for 30 ml at 65° C. Blots are developed with alkaline phosphatase-conjugated anti-digoxygenin and calorimetric substrate (Boehringer Mannheim) according to the manufacturer's instructions.

Phenotypic analysis of the rib-APP recombinants is performed as follows. Whole cell lysates, TCA-precipitated culture supernatants, and polysaccharide preparations are analyzed on silver stained SDS-PAGE and on immunoblots developed with convalescent swine sera to determine whether there are differences in protein, LPS, extracellular toxin, or capsular polysaccharide pr files between wild type AP100, AP225, and the rib-APP recombinants which are either V-factor independent nd nalidixic acid resistant or V-factor independent. Briefly, whole cell lysates and supernatants of AP100, AP225 (nalidixic acid resistant), APP (V-factor independent, Rib-) are prepared from overnight cultures grown in HIV+5 mM CaCl₂+appropriate antibiotic. Cells are separated by microcentrifugation and resuspended in SDS-PAGE sample buffer (Laemmli, Nature 22w: 680-685 (1970)). The culture supernatant is precipitated with an equal volume of 20% trichloroacetic acid (TCA) and resuspended in SDS-PAGE (sodium dodecyl sulfate-polyacrylamide gel electrophoresis) sample buffer. Cellular polysaccharides, including liopolysaccharide (LPS) and capsular polysaccharide, are prepared according to the cell lysis/proteinase K method of Kimura et al. (Infect. Immun. 51: 69-79 (1986)). All samples are analyzed on a 0.125% SDS-12% acrylamide gel using a discontinuous buffer system (Laemmli, Nature 227: 680-685 (1970)). Samples are transferred to nitrocellulose according to standard protocols (Sambrook et al. (Eds.). In: Molecular Cloning: A Laboratory Manual, 2nd. ed. Cold Spring Harbor Press, Cold Spring Harbor, N.Y. (1989)) and probed with convalescent serum from a pig infected with A. pleuropneumoniae serotype 1. Antigen-antibody complexes are detected with horseradish peroxidase-conjugated protein A (Boehringer Mannheim) and the calorimetric substrate 4-chloro-naphthol (BioRad, Hercules, Calif.). Production of serotype-specific capsular polysaccharide is measured by co-agglutination assay using hyper-immune rabbit anti-sera complexed to Staphylococcus aureus whole cells (Jolie et al., Vet. Microbiol. 38: 329-349 (1994)). APP rib-recombinants, which are either V-factor independent and nalidixic acid resistant or V-factor independent, do not have protein, LPS, extracellular toxin, or capsular polysaccharide profiles that substantially differ from that of the parent APP.

Attenuation of the rib-APP recombinants is confirmed by testing in anima as taught in U.S. Pat. No. 5,925,354 to Fuller et al. Briefly, six groups of three pigs each which are infected as follows: Group (1), 1 LD₅₀ (5×10⁶ cfu) of WT APP; Group (2), rib-APP recombinant at a dose 4 times the WT APP LD(,; Group (3), rib-APP recombinant at a dos 20 times the WT APP LD₅₀; Group (4), rib-APP recombinant at a dose 100 times the WT APP LD₅₀; Group (5), rib-APP recombinant at a dose 500 times the WT APP LD₅₀, and Group (6), rib-APP recombinant at WT APP dose ad complemented pTF76, which contains the intact riboflavin biosynthesis operon.

The pigs are monitored every four hours post-infection and scored or clinical signs of pleuropneumonia, including increased respiration rate and temperature; dyspnea; loss of appetite; and change in activity or attitude (depression) (Jolie et al., Vet. Microbiol. 45: 383-391 (1995)). Seriously ill animals, as determined by dyspnea and depression scores, are euthanized and necropsied immediately. Survivors are euthanized three days post-infection. All animals are necropsied and the lungs examined macroscopically for signs of A. pleuropneumoniae lesions. Severity and type of lesions are scored using a standard formula. Representative lung samples are collected for histopathology and bacteria culture. Attenuation is assessed as decreased mortality, decreased lung lesions scores, and/or decreased severity of clinical scores in comparison to the group infected with WT APP (Jolie et al., Vet. Microbiol. 45: 383-391 (1995)).

The rib-APP recombinants are tested as live avirulent vaccines against disease cause by A. pleuropneumoniae, using previously established procedures (Fuller et al., Vaccine 18: 2867-2877 (2000)). As shown in U.S. Pat. No. 5,925,354 to Fuller et al. and Fuller et al., Vaccine 18: 2867-2877 (2000), rib-APP recombinant containing the kanamycin gene was attenuated and was efficacious in vaccine challenge trials. The rib-APP recombinant made herein using nadV gene instead of the kanamycin gene for selection of the rib-APP recombinant is expected to be no less attenuated and efficacious than the rib-APP recombinant with the kanamycin gene.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

40. A method for immunizing a host against a Pasteurellaceae spp. comprising:
administering to the host an effective dose of a vaccine comprising a recombinant V-factor independent Pasteurellaceae spp. comprising a gene encoding nicotinamide phosphoribosyl transferase (NadV) inserted into a genomic nucleic acid sequence of a V-factor dependent Pasteurellaceae spp. or strain wherein the gene encoding the NadV disrupts expression of one or more genes encoded by the genomic nucleic acid sequence to produce the recombinant V-factor independent Pasteurellaceae spp. or strain, in a pharmaceutically acceptable carrier.

41. The method of claim 40, wherein the V-factor dependent Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

42. The method of claim 40, wherein the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp.

43. The method of claim 40, wherein the gene encoding the NadV is from Haemophilus ducreyi deposited as ATCC 27722.

44. The method of claim 40, wherein the gene encoding the NadV is operably linked to a heterologous promoter.

45. The method of claim 40, wherein the gene encoding the NadV comprises a nucleotide sequence with the nucleic acid sequence set forth in SEQ ID NO:1.

46. The method of claim 40, wherein the genomic nucleic acid sequence comprises one or more genes that are necessary for survival of the Pasteurellaceae spp. in vivo.

47. The method of claim 40, wherein the genomic nucleic acid sequence comprises one or more genes selected from the group consisting of genes for riboflavin biosynthesis, genes for aromatic amino acid biosynthesis, genes for isoleucine and valine biosynthesis, genes for a virulence factor, and combinations thereof.

48. The method of claim 40, wherein the genomic nucleic acid sequence encodes a gene selected from the group consisting of ribA, ribB, ribH, aroA, ilvI, lktC, apxIV, and combinations thereof.

49. The method of claim 40, wherein the vaccine contains an adjuvant.

50. The method of claim 40, wherein the recombinant Pasteurellaceae spp. is live.

51. The method of claim 40, wherein the recombinant Pasteurellaceae spp. is inactivated.

52. A method for reducing the cost of growing a V-factor dependent Pasteurellaceae spp. comprising:
(a) transforming the V-factor dependent Pasteurellaceae spp. with a gene encoding a nicotinamide phosphoribosyl transferase (NadV) to produce a recombinant Pasteurellaceae spp. wherein the gene encoding the NadV, which renders the V-factor dependent Pasteurellaceae spp. V-factor independent, is inserted into the genome of the V-factor dependent pasteurellaceae spp.; and
(b) growing the recombinant Pasteurellaceae spp. in media free of nicotinamide adenine dinucleotide (NAD) and nicotinamide mononucleotide (NMN) which reduces the cost of growing the V-factor dependent Pasteurellaceae spp.

53. The method of claim 52, wherein the Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

54. The method of claim 52, wherein the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp.

55. The method of claim 52, wherein the gene encoding the NadV is from Haemophilus ducreyi deposited as ATCC 27722.

56. The method of claim 52, wherein the gene encoding the NadV is operably linked to a heterologous promoter.

57. The method of claim 52, wherein the gene encoding the NadV comprises a nucleic acid sequence with the nucleic acid sequence set forth in SEQ ID NO:1.

58. The method of claim 52, wherein the gene encoding the NadV is on a plasmid.

59. The method of claim 52, wherein the gene encoding the NadV replaces a portion of a genomic nucleic acid sequence of the V-dependent Pasteurellaceae spp.

60. The method of claim 59, wherein the genomic nucleic acid sequence encodes one or more genes necessary for survival of the Pasteurellaceae spp. in vivo.

61. The method of claim 60, wherein the genomic nucleic acid sequence encodes one or more genes selected from the group consisting of genes for riboflavin biosynthesis, genes for aromatic amino acid biosynthesis, genes for isoleucine and valine biosynthesis, genes for a virulence factor, and combinations thereof.

62. The method of claim 60, wherein the genomic nucleic acid sequence encodes a gene selected from the group consisting of ribA, ribB, ribH, aroA, ilvI, lktC, apxIV, and combinations thereof.

63. A method for growing a V-factor dependent Pasteurellaceae spp. in a medium free of nicotinamide adenine dinucleotide (NAD) and nicotinamide mononucleotide (NMN) comprising:
(a) transforming the V-factor dependent Pasteurellaceae spp. with a gene encoding a nicotinamide phosphoribosyl transferase (NadV) to produce a recombinant Pasteurellaceae spp. wherein the gene encoding the NadV, which renders the V-factor dependent Pasteurellaceae spp. V-factor independent, is inserted into the genome of the V-factor dependent pasteurellaceae spp.; and
(b) growing the recombinant Pasteurellaceae spp. in the medium free of NAD and NMN which reduces the cost of growing the V-factor dependent Pasteurellaceae spp.

64. The method of claim 63, wherein the Pasteurellaceae spp. is selected from the group consisting of Actinobacillus pleuropneumoniae, Actinobacillus suis, Haemophilus influenzae, Haemophilus paragallinarum, Haemophilus parainfluenzae, Haemophilus parasuis, Haemophilus ducreyi.

65. The method of claim 63, wherein the gene encoding the NadV is from a bacterium selected from the group consisting of Actinobacillus actinomycetemcomitans, Actinobacillus lignieresii, Actinobacillus pleuropneumoniae, Actinobacillus suis, Deinococcus radiodurans, Haemophilus aphrophilus, Haemophilus ducreyi, Haemophilus haemoglobinophilus, Haemophilus influenzae, Haemophilus ovis, Haemophilus paragallinarum, Haemophilus parainfluenzae, Haemophilus parasuis, Haemophilus somnus, Mycoplasma genitalium, Mycoplasma pneumoniae, Pasteurella haemolytica, Pasteurella multocida, Shewanella putrefaciens, and Synechocystis spp.

66. The method of claim 63, wherein the gene encoding the NadV is from Haemophilus ducreyi deposited as ATCC 27722.

67. The method of claim 63, wherein the gene encoding the NadV is operably linked to a heterologous promoter.

68. The method of claim 63, wherein the gene encoding the NadV comprises a nucleic acid sequence with the nucleic acid sequence set forth in SEQ ID NO:1.

69. The method of claim 63, wherein the gene encoding the NadV is on a plasmid.

70. The method of claim 63, wherein the gene encoding the NadV replaces a portion of a genomic nucleic acid sequence of the V-dependent Pasteurellaceae spp.

71. The method of claim 70, wherein the genomic nucleic acid sequence encodes one or more genes necessary for survival of the Pasteurellaceae spp. in vivo.

72. The method of claim 71, wherein the genomic nucleic acid sequence encodes one or more genes selected from the group consisting of genes for riboflavin biosynthesis, genes for aromatic amino acid biosynthesis, genes for isoleucine and valine biosynthesis, genes for a virulence factor, and combinations thereof.

73. The method of claim 71, wherein the genomic nucleic acid sequence encodes a gene selected from the group consisting of ribA, ribB, ribH, aroA, ilvI, lktC, apxIV, and combinations thereof.

74. The method of claim 9, 40, 52, or 63, wherein the NadV comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

75. The genetically defined mutant of claim 19, wherein the NadV comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

76. The vaccine of claim 28, wherein the NadV comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

77. An isolated nucleic acid which encodes a protein that confers V-factor independence to a V-factor dependent bacteria when transformed into the V-factor dependent bacteria selected from the group consisting of SEQ ID NO:1, SEQ ID NO:13, SEQ ID NO:14, SEQ ID NO:15, SEQ ID NO:16, SEQ ID NO:17, and SEQ ID NO:19.
